(19) United States
(12) Patent Application Publication
(10) Pub. No.: US 2017/0191125 A1
(43) Pub. Date: Jul. 6, 2017

(54) SEQUENCING DEVICE

(71) Applicant: Omniome, Inc., San Diego, CA (US)

(72) Inventors: Kandaswamy Vijayan, San Diego, CA (US); Maxim Abashin, San Diego, CA (US); Yi Zhang, San Diego, CA (US); Espir Kahatt, San Diego, CA (US); Kerry Wilson, San Diego, CA (US)

(73) Assignee: Omniome, Inc., San Diego, CA (US)

(21) Appl. No.: 15/392,862

(22) Filed: Dec. 28, 2016

Related U.S. Application Data
(60) Provisional application No. 62/273,346, filed on Dec. 30, 2015.

(51) Int. Cl. C12Q 1/68; G01N 21/64

(52) U.S. Cl. CPC C12Q 1/6869; G01N 21/6486

(57) ABSTRACT

Systems and methods for performing DNA sequencing. An example system includes a flow cell, a mechanism to generate fluid flow, a number of reservoirs for containing respective fluids, and a number valves configured such that fluid from any particular one of the plurality of reservoirs can be individually supplied to the flow cell under the impetus of the mechanism to generate fluid flow by opening of the respective valve of the particular reservoir and closing the other valves. Fluids containing test nucleotides may be sequentially flowed through the flow cell and the flow cell imaged at each step to detect binding of the test nucleotides to a sample. The nucleotide sequence of the sample is derived from the images. The sample may be arrayed on a sensing surface of a prism, and the images may be obtained, for example, by surface plasmon resonance imaging (SPRi) of the sensing surface or other techniques.

### CROSS-REFERENCE TO RELATED APPLICATIONS

This application claims priority to U.S. Provisional Application No. 62/273,346, filed on Dec. 30, 2015, which is hereby incorporated by reference in its entirety.

### BACKGROUND OF THE INVENTION

The determination of nucleic acid sequence information is important in biological and medical research. The process of determining sequence information is commonly called “sequencing.” The sequence information is helpful for identifying gene associations with diseases and phenotypes, identifying potential drug targets, and understanding the mechanisms of disease development and progress. Sequence information is an important part of personalized medicine, where it can be used to optimize the diagnosis, treatment, or prevention of disease in a specific subject.

Given the wide applicability and utility of nucleic acid sequence information, improved systems and methods for sequencing are desired, for example to reduce the cost of obtaining sequence information.

### BRIEF SUMMARY OF THE INVENTION

Embodiments of the invention provide systems and methods for nucleic acid sequencing.

According to one aspect, a system includes a computerized controller, and a prism having an input face, an output face, and a detection face. The system further includes a flow cell disposed adjacent the detection face of the prism, a plurality of reservoirs for holding respective fluids, a plurality of valves connected respectively with the plurality of reservoirs, a mechanism to generate fluid flow, an illumination system positioned to direct light into the input face of the prism such that the light reaches the detection face of the prism, and a sensing system positioned to image a plane in or adjacent the flow cell. The reservoirs, valves, mechanism to generate fluid flow, and flow cell are configured such that fluid from any particular one of the plurality of reservoirs can be individually supplied to the flow cell under the impetus of the mechanism to generate fluid flow and under control of the computerized controller, by opening of the respective valve of the particular reservoir and closing the other valves.

### BRIEF DESCRIPTION OF THE DRAWINGS

FIG. 1 illustrates a block diagram of a system in accordance with embodiments of the invention.

FIG. 2 illustrates nanoballs bound to grid locations of a flow cell, in accordance with embodiments of the invention.

FIG. 3 illustrates an example arrangement of a flow cell, an illumination system, and a sensing system, in accordance with embodiments of the invention.

FIG. 4 illustrates another example arrangement of a flow cell, an illumination system, and a sensing system, in accordance with embodiments of the invention.

FIG. 5 illustrates a system in accordance with other embodiments of the invention.

FIGS. 6A and 6B illustrate two trapezoidal prisms in accordance with embodiments of the invention.

FIG. 7A illustrates a flow cell cavity arrangement in accordance with embodiments of the invention, and FIG. 7B illustrates the flow of fluids through the flow cell arrangement of FIG. 7A.

FIG. 8A illustrates a flow cell cavity arrangement in accordance with embodiments of the invention, and FIG. 8B illustrates the flow of fluids through the flow cell arrangement of FIG. 8A.

FIG. 9A illustrates a flow cell cavity arrangement in accordance with embodiments of the invention, and FIG. 9B illustrates the flow of fluids through the flow cell arrangement of FIG. 9A.

FIG. 10 illustrates an instrument in accordance with embodiments of the invention.

FIGS. 11A and 11B illustrate the instrument of FIG. 10 with its cover removed.

FIG. 12 illustrates a disposable fluidic interface of the instrument of FIG. 10 in isolation.

FIG. 13 is an exploded view of the disposable fluidic interface of FIG. 12.

FIG. 14A illustrates a cutaway view of the disposable fluidic interface of FIG. 12, showing valves in their normally closed positions.

FIG. 14B shows a valve of FIG. 14A, in its open position.

FIG. 15 illustrates a cartridge according to embodiments of the invention.

FIG. 16A shows an unprocessed surface plasmon resonance (SPR) image of a microspotted array on a gold thin film, in accordance with embodiments of the invention.

FIG. 16B illustrates a processed SPR image with background subtraction prior to exposure to sequencing reagents, in accordance with embodiments of the invention.

FIG. 16C shows the change in relative reflected intensity on the microspotted chip of FIG. 16B after exposure to sequencing reagents.

FIG. 17A shows an SPR image of the flow patterned chip in accordance with embodiments of the invention.

FIG. 17B shows raw sequencing data collected from a region of the phiX bacteriophage genome, in accordance with embodiments of the invention.

FIG. 17C shows the resulting positive and negative base calls derived from the raw data of FIG. 17B.

FIG. 18 schematically illustrates nanohole sensing, in accordance with embodiments of the invention.

FIG. 19 illustrates a module including the nanohole sensing system of FIG. 18, in accordance with embodiments of the invention.

FIG. 20 illustrates a sensogram recorded using nanohole sensing, in accordance with embodiments of the invention.

FIG. 21 illustrates another sensing modality usable in embodiments of the invention, configured for utilizing grating waveguide resonance.

FIGS. 22A and 22B illustrate the effect of grating-waveguide resonance, in accordance with embodiments of the invention.

FIG. 23 illustrates images of a flow-patterned substrate taken using grating-waveguide resonance (GWR), in accordance with embodiments of the invention.

FIG. 24 illustrates averaged intensity readings taken from a polydopamine (universal) surface chemistry using GWR, in accordance with embodiments of the invention.

FIG. 25 shows a grating used for enhancement of fluorescence, in accordance with embodiments of the invention.

FIG. 26 shows a test system in accordance with embodiments of the invention.

FIG. 27A illustrates a digital image taken with the system of FIG. 26.

FIG. 27B illustrates another digital image taken with the system of FIG. 26.

FIG. 27C illustrates a digital slice taken through a portion of the image of FIG. 27B.

FIG. 28A illustrates a digital slice taken through a portion of an image taken using total internal reflectance fluorescence (TIRF) imaging, in the area of a particular nanoball.

FIG. 28B illustrates a digital slice taken through a portion of an image taken using surface plasmon enhanced fluorescence (SPEF) imaging, in the area of a particular nanoball.

### DETAILED DESCRIPTION

The present disclosure provides a device that can be used for a variety of molecular analyses, such as nucleic acid sequencing. In some embodiments, sequencing is carried out as described in commonly owned U.S. patent application Ser. No. 14/805,381, which is incorporated by reference herein in its entirety. Briefly, methods for determining the sequence of a template nucleic acid molecule can be based on a repetitive process wherein each cycle in the process provides information toward identifying one or more nucleotides in a target nucleic acid. The sum of the information from the cycles provides the sequence of nucleotides for the target nucleic acid. In particularly useful sequencing protocols each cycle is carried out by forming a ternary complex (between polymerase, primed nucleic acid and cognate nucleotide) under specified conditions. The method can generally include a step of examining the ternary complex prior to a correct nucleotide being incorporated into the nucleic acid by covalent attachment to the 3′ end of the primer. For example, the method can involve providing a template nucleic acid molecule primed with a primer; contacting the primed template nucleic acid molecule with a first reaction mixture that includes a polymerase and at least one nucleotide molecule; detecting interaction of the polymerase and nucleotide with the primed template nucleic acid molecule, without covalent incorporation of the nucleotide molecule into the primed template nucleic acid; and identifying a next base in the template nucleic acid using the detected interaction of the polymerase and nucleotide with the primed template nucleic acid molecule. In this procedure, ternary complex stabilization advantageously enhances discrimination between correct and incorrect nucleotides.

In particular embodiments, a device of the present disclosure can detect ternary complexes formed at each cycle of a sequencing process without the need for exogenous labels on one or more of the reactants that would typically be labeled when carrying out a sequencing process on other detection platforms. For example, the sequencing reaction can be performed using polymerase, nucleotides and primed nucleic acids that all lack exogenous labels that are used for detection. However, in some embodiments of the present disclosure the polymerase can be labeled with an exogenous moiety. Alternatively or additionally to polymerase labeling, the nucleotides can be labeled.

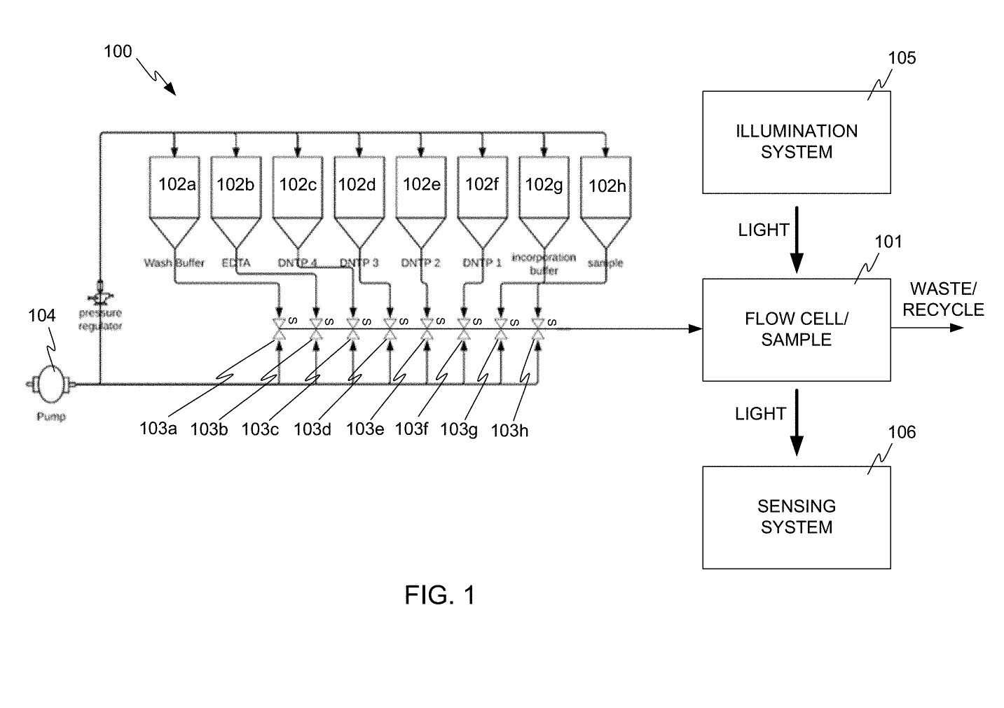

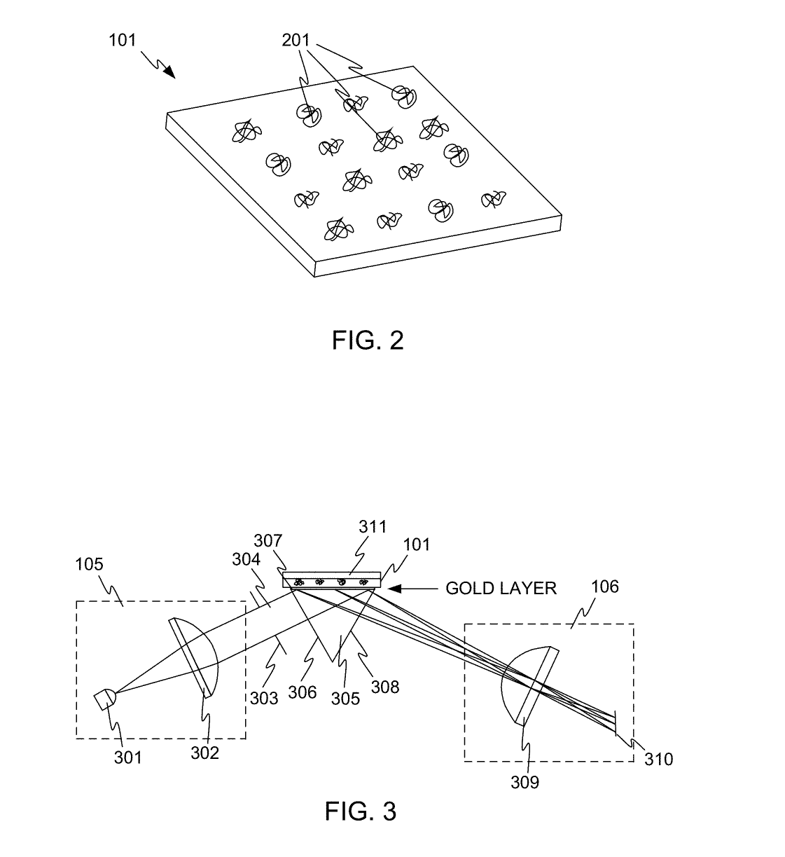

FIG. 1 illustrates a block diagram of a system 100 in accordance with embodiments of the invention. System 100 includes a flow cell 101, in which a sample of material to be sequenced can be placed. For example, the sample may be an array of “nanoballs” 201 of amplified DNA fragments, bound to a grid of locations within flow cell 101, as shown in FIG. 2. While only a few nanoballs 201 are shown in FIG. 2 for ease of explanation, more or fewer may be present. Depending on the target application, many, many receptors may be present within flow cell 101, for example up to millions, tens of millions, or more. DNA nanoballs can be made using methods and compositions as described, for example, in U.S. Pat. No. 7,910,354; or US Pat. App. Publ. Nos. 2009/0264299 A1, 2009/0011943 A1, 2009/0005252 A1, 2009/0155781 A1, or 2009/0118488 A1; or Drmanac et al., 2010, Science 327(5961): 78-81; each of which is incorporated herein by reference.

Nanoballs are one type of nucleic acid amplification product that can be used to form a feature on an array. Other useful amplification products include those produced by solid-phase amplification techniques. For example, amplification can be carried out using bridge amplification to form nucleic acid clusters on a surface. Useful bridge amplification methods are described, for example, in U.S. Pat. Nos. 5,641,658 or 7,115,400; or U.S. Pat. App. Pub. Nos. 2002/0055100 A1, 2004/0096853 A1, 2004/0002090 A1, 2007/0128624 A1; or 2008/0009420 A1, each of which is incorporated herein by reference. Another useful method for amplifying nucleic acids on a surface is rolling circle amplification (RCA), for example, as described in Lizardi et al., Nat. Genet. 19:225-232 (1998) and U.S. Pat. App. Pub. No. 2007/0099208 A1, each of which is incorporated herein by reference. Emulsion PCR on beads can also be used, for example as described in Dressman et al., Proc. Natl. Acad. Sci. USA 100:8817-8822 (2003), WO 05/010145, US Pat. App. Pub. No. 2005/0130173 A1 or U.S. Pat. App. Pub. No. 2005/0064460 A1, each of which is incorporated herein by reference. A system or method of the present disclosure can use one or more of the reagents described in the above references for making and using nanoballs or other nucleic acid features.

Referring again to FIG. 1, system 100 also includes a number of reservoirs 102a-102h (collectively reservoirs 102), for holding various buffers, nucleotides, and other fluids. While eight reservoirs are shown in the example of FIG. 1, more or fewer reservoirs may be present in other embodiments. The reservoirs can contain reagents used for creating nucleic acid features and/or reagents for sequencing nucleic acids such as those set forth herein or in references incorporated by reference herein.

Each of reservoirs 102a-102h is connected to respective valve 103a-103h (collectively valves 103), such that under the control of a computerized controller (not shown), fluid from any one of reservoirs 102 can be individually supplied to flow cell 101 under the impetus of a mechanism to generate fluid flow, for example pump 104. In some embodiments, pump 104 or other mechanism to generate fluid flow may produce a constant fluid flow, and in other embodiments, may produce a variable fluid flow. Flow cell 101 can be illuminated by an illumination system 105 and optically sensed by a sensing system 106. Valves 103 may be arranged either serially, in parallel, or in any combination of configurations. In some embodiments, valves 103 are arranged serially to prevent pockets of reagent that could contaminate subsequent steps of the sequencing reaction. The wash buffer is situated at the position furthest from the flow cell to ensure that all reagents are thoroughly washed from the channel and flow cell prior to subsequent sequencing steps. Valves 103 may be actuated by pneumatic, mechanical, or electrical means.

Sensing system 106 may be, for example, a digital camera having an array light sensor. Various optical devices such as prisms, lenses, filters, and the like may be present between flow cell 101 and sensing system 106, as is explained in more detail below. It should be recognized that FIG. 1 is highly schematic, and is not intended to represent specific component arrangements. Some specific arrangements are described below.

In a basic manner of operation of system 100, a sample to be sequenced is placed in flow cell 101. The sample may be previously prepared such that nucleic acid features are present on the surface prior to introducing the flow cell to the system. Alternatively, the nucleic acid features may be constructed in part by system 100 for example, by a solid phase amplification technique set forth herein or known in the art. Once the sample is in place, a test nucleotide may be delivered to flow cell 101, for example from reservoir 102c. In the presence of polymerase, the test nucleotide binds to sites in the sample having cognate nucleotide positions adjacent to the 3′ end of a primer (i.e. the test nucleotide occupies the position of the “next correct” nucleotide for primer extension). The binding creates changes in the sites that are detectable by sensing system 106. Depending on the sensing technology being used, the detectable change may be a change in apparent reflectance due to surface plasmon resonance, may be the presence of a fluorescent marker supplied with the nucleotide or polymerase, may be the presence of fluorescence excited by illumination system 105 without the need for a marker, or may be some other kind of detectable change caused by binding between a primed nucleic acid, polymerase and nucleotide to form a ternary complex. A number of sensing technologies that may be used in embodiments of the invention are described in U.S. patent application Ser. No. 14/805,381 filed Jul. 21, 2015 and titled “Nucleic Acid Sequencing Methods and Systems”, the entire disclosure of which is hereby incorporated by reference herein for all purposes.

Sensing system 106 then detects the changes in the sample resulting from the introduction of the test nucleotide, for example by taking an image of the area of the flow cell and analyzing the digital image to detect the locations of any changes. The changes indicate the locations at which the supplied test nucleotide attached to the sample via ternary complex formation. Because the type of the test nucleotide is known, the nucleotide to which it attached is inferable, being the complementary nucleotide of the test nucleotide.

Preferably, flow cell 101 is washed to remove any unattached reagents such as nucleotides, and a second test reagent (e.g. second type of nucleotide) is supplied to flow cell 101, for example from reservoir 102d. Any changes to the sample are detected in a similar manner, and locations where binding of the second test nucleotide (e.g. via formation of a ternary complex) are detected are noted as containing primed nucleic acids having a sequence position that is complementary to the second test nucleotide.

This process is repeated so that the nucleotide sequence at each sample location is cumulatively determined.

The above description is highly simplified, and is presented in the interest of assisting in the understanding of the specific embodiments described below. More detail about the sequencing process may be found below or in U.S. patent application Ser. No. 14/805,381, which is incorporated herein by reference.

Although the present disclosure exemplifies several aspects of the systems and methods set forth herein in the context of nucleic acid sequencing, it will be understood that a variety of other analytes can be detected. Analytes that participate in binding interactions with probes that can be attached to a surface are particularly useful. Similarly, binding assays that have been, or can be, modified to occur on solid-phase supports are also useful. Exemplary analytes that can be detected include, but are not limited to, biological macromolecules such as proteins, enzymes, receptors, antibodies, polysaccharides or the like; analogs of biological macromolecules such as nucleic acid analogs (e.g. protein nucleic acid), antibody analogs (e.g. Fab or F(ab′)₂), mutant enzymes that retain binding affinity for substrates or the like; biological particles such as cells, viruses, vesicles, nanopores, ribosomes, organelles, nuclei or the like; biological small molecules such as metabolites, saccharides, amino acids, nucleotides, enzyme cofactors, or analogs thereof; or synthetic analytes such as candidate ligands for target receptors, candidate therapeutic agents such as enzyme inhibitors, nanoparticles, beads or the like. Particularly useful binding assays include, but are not limited to, immunosorbent assays which can be performed without the need for enzyme labels or other labels that are typically used in ELISA formats, receptor-ligand binding assays, cell surface receptor biding assays, nucleic acid hybridization assays, ribosome binding assays, protein-protein binding assays or the like.

An advantage of the systems and methods set forth herein is that a variety of different types of binding assays can be run on the same system. This is possible in many embodiments due to localized detection of different binding events at discrete surface features and lack of unwanted background signal from target analytes that remain in solution. When using a system of the present disclosure, different types of probe analytes can be attached to discrete features on a surface, the location of the probe analytes can be known or determined, and different target analytes can be delivered in solution under conditions that allow them to bind to probes for which they have an affinity. The different binding assays can be run on the same substrate either sequentially or simultaneously (i.e. in parallel).

Optical Systems

FIG. 3 illustrates an example arrangement of flow cell 101, illumination system 105, and sensing system 106 in more detail, in accordance with embodiments of the invention. In illumination system 105, a light source 301 emits light which is captured and sufficiently collimated by a lens 302. In some embodiments, light source 301 may be a light emitting diode or an array of light emitting diodes emitting light at a wavelength of about 650 nm, but other wavelengths may be used on other embodiments, and other kinds of light sources may be used. For example, a laser with beam expanding optics may be used. The light produced by illumination system 105 may be coherent or non-coherent. In some embodiments, multiple light emitting diodes or lasers may be used emitting light in different wavelengths.

In some embodiments, the light source emits a narrow range of wavelengths (less than 10 nm full width at half maximum) centered on a wavelength in the visible light spectrum. In other embodiments, the light source emits a broad range of wavelengths onto a sample at a fixed angle, and the reflected light is then dispersed by a diffraction grating onto a CCD or linear photodiode array to determine the resonant wavelength. In some embodiments, one or more optical filters may be used to narrow the wavelength content of the illumination light.

Lens 302 may be a simple plano convex element having a focal length of about 24 mm, or may be a more complex lens such as a multi-element lens. Other focal lengths may also be used in other embodiments. Preferably, an aperture 303 limits the size of the illumination beam 304. The size of aperture 303 may be selected in accordance with the capabilities of the particular embodiment, but in one example, aperture 303 may have a diameter of about 10 mm.

Beam 304 enters a prism 305 through an input face 306. Prism 305 may be a simple triangular prism made of F2 glass or another suitable glass. In other embodiments, prism 305 may be molded from a polymer such as polycarbonate or another suitable clear polymer.

Flow cell 101 is positioned on a top or detection face 307 of prism 305. A cover glass 311 may also be present over flow cell 101, opposite detection face 307 of prism 305. In some embodiments, detection surface 307 is coated with a thin layer of gold, silver, aluminum, or another suitable material. The prism can be an integral component of the flow cell such that the coating is directly on a surface of the prism and reagents flow over the surface of the prism when flowing through the flow cell. In other embodiments, an optically transparent window of a flow cell having the coating is coupled with the prism. As such, the prism can be an integral part of a flow cell or the prism can be a separate component that is removably coupled with a window of the flow cell.

Illumination system 105 preferably produces plane polarized light (p-polarization, where the electric field of the incident photon has a component normal to the plane of the gold film), which is then passed through one face of the prism at a defined angle wherein some of the light is absorbed by the gold film. Another portion of beam 304 reflects from detection surface 307, either by total internal reflection or by reflection from the metal coating on detection surface 307. Imaging system 106 images an area of detection surface 307 through output face 308 of prism 305. Imaging system 106 includes a lens 308, which may be a simple plano convex or aspheric singlet having a focal length of about 24 mm, although other kinds of lenses may be used.

Lens 309 forms an image on an electronic array light sensor 310. Sensor 310 may be, for example, a complementary metal oxide semiconductor (CMOS) sensor, a charge coupled device (CCD) sensor, or another kind of sensor having a number of light sensitive areas called pixels arranged in an array. Sensor 310 may be part of a camera, for example a DMM24UJ003 board camera available from The Imaging Source of Bremen, Germany. In any event, sensor 310 produces signals indicating the intensity of light received from the locations on detection face 307 corresponding to the sensor pixels. These signals may be compiled into a digital image. Some of the digital image may correspond to one or more reference regions to account for changes in background signal due to bulk refractive index changes, nonspecific binding of soluble factors, thermal fluctuations, changes in the surface of the sensing element, or other effects.

As is shown in FIG. 3, the plane of sensor 310 may be oblique to the optical axis of sensing system 106, in order to correctly image detection face 307, which is also at an oblique angle.

Optionally, detecting a change in refractive index is accomplished in one or a combination of means, including, but not limited to, surface plasmon resonance sensing, localized plasmon resonance sensing, plasmon-photon coupling sensing, transmission sensing through sub-wavelength nanoholes (enhanced optical transmission), photonic crystal sensing, interferometry sensing, refraction sensing, guided mode resonance sensing, ring resonator sensing, or ellipsometry sensing. Optionally, probe analytes can be localized to features on the surface and target analytes can be delivered under conditions wherein the probes and targets interact such that a change in local refractive index can be detected and used to identify or characterize the interaction. For example, nucleic acid molecules may be localized to a surface, wherein the interaction of polymerase with nucleic acids in the presence of various nucleotides may be measured as a change in the local refractive index.

Optionally, a probe analyte (e.g. a template nucleic acid) is tethered to or localized appropriately on or near a surface, such that the interaction of the target analyte (e.g. interaction of polymerase and template nucleic acid in the presence of nucleotides) changes the light transmitted across or reflected from the surface. The surface may contain nanostructures. Optionally, the surface is capable of sustaining plasmons or plasmon resonance. Optionally, the surface is a photonic substrate, not limited to a resonant cavity, resonant ring or photonic crystal slab. Optionally, the surface is a guided mode resonance sensor. Optionally, the probe analyte (e.g. nucleic acid) is tethered to, or localized appropriately on or near a nanohole array, a nanoparticle or a microparticle, such that the interaction of target analyte (e.g. interaction of polymerase and template nucleic acid in the presence of nucleotides) changes the absorbance, scattering, reflection or resonance of the light interacting with the microparticle or nanoparticle.

Optionally, extraordinary optical transmission (EOT) through a nanohole array may be used to monitor probe/target (e.g. nucleic-acid/polymerase) interactions. Light transmitted across subwavelength nanoholes in plasmonic metal films is higher than expected from classical electromagnetic theory. This enhanced optical transmission may be explained by considering plasmonic resonant coupling to the incident radiation, whereby at resonant wavelength, a larger than anticipated fraction of light is transmitted across the metallic nanoholes. The enhanced optical transmission is dependent on the dimensions and pitch of the nanoholes, properties of the metal, as well as the dielectric properties of the medium on either side of the metal film bearing the nanoholes. In the context of a biosensor, the transmissivity of the metallic nanohole array depends on the refractive index of the medium contacting the metal film, whereby, for instance, the interaction of polymerase with nucleic acid attached to the metal surface may be monitored as a change in intensity of light transmitted across the nanoholes array. The elegance of the EOT/plasmonic nanohole array approach is that the instrumentation and alignment requirements of surface plasmon resonance may be replaced by very compact optics and imaging elements. For instance, just a low power LED illumination and inexpensive CMOS or CCD camera may suffice to implement robust EOT plasmonic sensors. An exemplary nanohole array-based surface plasmon resonance sensing device is described in C. Escobedo et al., “Integrated Nanohole Array Surface Plasmon Resonance Sensing Device Using a Dual-Wavelength Source,” Journal of Micromechanics and Microengineering 21, no. 11 (Nov. 1, 2011): 115001, which is herein incorporated by reference in its entirety.

The plasmonic nanohole array may be patterned on an optically opaque layer of gold (greater than 50 nm thickness) deposited on a glass surface. Optionally, the plasmonic nanohole array may be patterned on an optically thick film of aluminum or silver deposited on glass. Optionally, the nanohole array is patterned on an optically thick metal layer deposited on low refractive index plastic. Patterning plasmonic nanohole arrays on low refractive index plastics enhances the sensitivity of the device to refractive index changes by better matching the refractive indices on the two sides of the metal layer. Optionally, refractive index sensitivity of the nanohole array is increased by increasing the distance between holes. Optionally, nanohole arrays are fabricated by replication, for example, by embossing, casting, imprint-lithography, or template-stripping. Optionally, nanohole arrays are fabricated by self-assembly using colloids. Optionally, nanohole arrays are fabricated by projection direct patterning, such as laser interference lithography.

A nano-bucket configuration may be preferable to a nanohole configuration. In the nanohole configuration, the bottom of the nano-feature is glass or plastic or other appropriate dielectric, whereas in the nano-bucket configuration, the bottom of the nano-feature comprises a plasmonic metal. The nano-bucket array configuration may be easier to fabricate in a mass production manner, while maintaining the transmission sensitivity to local refractive index.

Optionally, the nanohole array plasmonic sensing is combined with lens-free holographic imaging for large area imaging in an inexpensive manner. Optionally, a plasmonic biosensing platform comprises a plasmonic chip comprising nanohole arrays, a light-emitting diode source configured to illuminate the chip, and a CMOS imager chip to record diffraction patterns of the nanoholes, which is modulated by molecular binding events on the surface. The binding events may be the formation of a closed-complex between a polymerase and a template nucleic acid in the presence of a nucleotide.

The methods to functionalize surfaces (e.g. for nucleic acid attachment) for surface plasmon resonance sensing may be directly applied to EOT nanohole arrays as both sensing schemes employ similar metal surfaces to which probes, such as nucleic acids, can be attached.

Optionally, the refractive index changes associated with probe/target interaction may be detected or monitored on nanostructured surfaces that do not support plasmons. Optionally, guided mode resonance may be used to detect or monitor the probe/target interaction. Guided-mode resonance or waveguide-mode resonance is a phenomenon wherein the guided modes of an optical waveguide can be excited and simultaneously extracted by the introduction of a phase-matching element, such as a diffraction grating or prism. Such guided modes are also called “leaky modes”, as they do not remain guided, and have been observed in one and two-dimensional photonic crystal slabs. Guided mode resonance may be considered a coupling of a diffracted mode to a waveguide mode of two optical structured placed adjacent or on top of each other. For instance, for a diffraction grating placed on top of an optical waveguide, one of the diffracted modes may couple exactly into the guided mode of the optical waveguide, resulting in propagation of that mode along the waveguide. For off-resonance conditions, no light is coupled into the waveguide, so the structure may appear completely transparent (if dielectric waveguides are used). At resonance, the resonant wavelength is strongly coupled into the waveguide, and may be coupled out of the structure depending on downstream elements from the grating-waveguide interface. In cases where the grating coupler is extended over the entire surface of the waveguide, the light cannot be guided, as any light coupled in is coupled out at the next grating element. Therefore, in a grating waveguide structure, resonance is observed as a strong reflection peak, whereas the structure is transparent to off-resonance conditions. The resonance conditions are dependent on angle, grating properties, polarization and wavelength of incident light. For cases where the guided mode propagation is not present, for instance due to a grating couple to the entire surface of the waveguide, the resonant mode may also be called leaky-mode resonance, in light of the strong optical confinement and evanescent propagation of radiation in a transverse direction from the waveguide layer. Change in dielectric properties near the grating, for instance due to binding of biomolecules affects the coupling into the waveguide, thereby altering the resonant conditions. Optionally, where nucleic acid molecules are attached to the surface of grating waveguide structures, the polymerase/nucleic-acid interaction may be detected or monitored as a change in wavelength of the leaky mode resonance.

Optionally, a diffraction element may be used directly on a transparent substrate without an explicit need for a waveguide element. The change in resonance conditions due to probe/target interactions near the grating nanostructure may be monitored as resonant wavelength shifts in the reflected or transmitted radiation.

Optionally, reflected light from a probe-attached, guided mode resonant sensor may be used to detect or monitor the probe/target interaction. A broadband illumination source may be employed for illumination, and a spectroscopic examination of reflected light could reveal changes in local refractive index due to target binding.

Optionally, a broadband illumination may be used and the transmitted light may be examined to identify resonant shifts due to probe/target interaction. Optionally, a linearly polarized narrow band illumination may be used, and the transmitted light may be filtered through a cross-polarizer; wherein the transmitted light is completely attenuated due to the crossed polarizers excepting for the leaky mode response whose polarization is modified. This implementation converts refractive index detecting or monitoring to a simple transmission assay that may be monitored on inexpensive imaging systems. This exemplary embodiment is aided by published material that describe the assembly of the optical components, Yousef Nazirizadeh et al., “Low-Cost Label-Free Biosensors Using Photonic Crystals Embedded between Crossed Polarizers,” Optics Express 18, no. 18 (Aug. 30, 2010): 19120-28, which is incorporated herein in its entirety.

Alongside nanostructured surfaces, plain, un-structured surfaces may also be used advantageously for detecting or monitoring refractive index modulations resulting from probe/target interactions. Optionally, interferometry may be employed to detect or monitor the interaction of probe and target (e.g. interaction of polymerase with double stranded nucleic acid) bound to an un-structured, optically transparent substrate. Optionally, probe molecules may be attached to the top surface of a glass slide (by any means known in the art), and the system illuminated from the bottom surface of the glass slide. There are two reflection surfaces in this configuration, one reflection from the bottom surface of the glass slide, and the other from the top surface which has probes (e.g. nucleic acid molecules) attached to it. The two reflected waves may interfere with each other causing constructive or destructive interference based on the path length differences, with the wave reflected from the top surface modulated by the changes in dielectric constant due to the bound probes (and subsequently by the interaction of target with the bound probe). With the reflection from the bottom surface unchanged, any binding to the probe may be reflected in the phase difference between the beams reflected from the top and bottom surfaces, which in turn affects the interference pattern that is observed. Optionally, bio-layer interferometry is used to detect or monitor the probe/target interaction. Bio-layer interferometry may be performed on commercial devices such as those sold by Pall Forte Bio corporation.

The reflected light from the top surface can be selectively chosen by using focusing optics. The reflected light from the bottom surface is disregarded because it is not in the focal plane. Focusing only on the probe-attached top surface, the light collected by the focusing lens comprises a planar wave, corresponding to the partially reflected incident radiation, and a scattered wave, corresponding to the radiations scattered in the collection direction by molecules in the focal plane. These two components may be made to interfere if the incident radiation is coherent. This scattering based interferometric detection is extremely sensitive, and can be used to detect down to single protein molecules.

In some embodiments, system 100 detects the binding of analytes to the sample using the phenomenon of surface plasmon resonance (SPR). Surface plasmon resonance sensing is a method for the label-free detection of analytes such as proteins, enzymes, macromolecules, nucleic acids, nanoparticles, vesicles, cells, exosomes, organelles, or other analytes, due to their interaction with light impinging upon a thin gold film (the sensing element or detection surface) at a defined angle and wavelength. The interaction of light with the gold film induces a collective oscillation of electrons at the gold/environment interface that produces a highly sensitive evanescent field at the interface. The evanescent field is highly sensitive to perturbations in refractive index of the surrounding environment. In some embodiments, the formation of a ternary complex on a nucleic acid feature creates slight changes in the resonance conditions, and can be detected as changes in the apparent reflectivity of the gold layer on detection surface 307. In some embodiments, the angle of incidence of the illumination light with respect to detection surface 307 can be varied to measure changes in reflected intensity. In other embodiments, the angle of incidence remains fixed. In any event, changes in reflected intensity are measured as a sequencing reaction proceeds on nucleic acid features. The instrument can be configured in such a way that either increasing or decreasing intensity can correspond to the detection of a sequencing step.

By taking digital images of a sample array of nucleic acid features after each application of sequencing reagent (e.g. polymerase and test nucleotides), features where reflectivity changes have occurred can be detected and therefore the nucleic acid to which a particular nucleotide bound as the next correct nucleotide can be determined. For example one can determine which of nanoballs 201 contained a sequence to which the test nucleotide bound in a ternary complex.

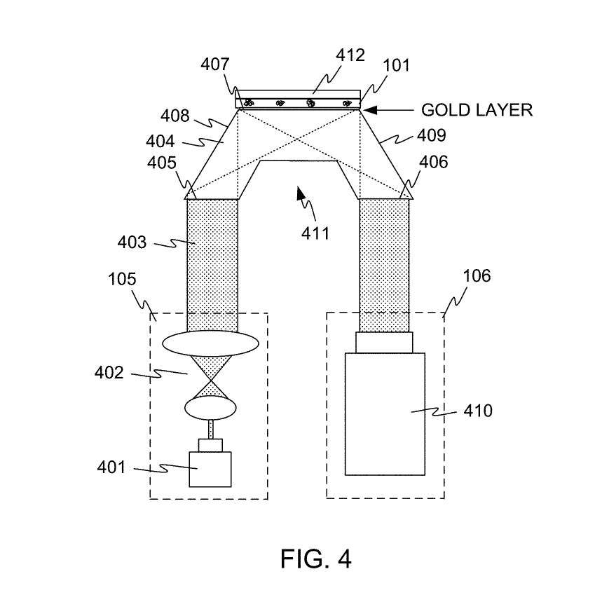

FIG. 4 illustrates a system 400 using another example arrangement of flow cell 101, illumination system 105 and sensing system 106, in accordance with other embodiments of the invention. In the example of FIG. 4, a laser 401 and beam expander 402 are used to create illumination beam 403. A trapezoidal prism 404 is used, rather than a triangular prism. Trapezoidal prism 404 includes an input surface 405 and an output surface 406 coplanar with input surface 405. A detection surface 407 is parallel to and spaced apart from input and output surfaces 405 and 406. A first angled surface 408 joins one edge of detection surface 407 and one edge of input surface 405, and a second angled surface 409 joins the other edge of detection surface 407 with an edge of output surface 406. Prism 404 may be made of any suitable material, for example F2 or another glass, or poly(methyl methacrylate) (PMMA) or another suitable polymer. As is shown in the embodiment shown in FIG. 4, a portion 411 of prism 404 is removed, for example to facilitate making prism 404 by injection molding of a polymer. In other embodiments, this portion need not be absent, and input and output faces 405 and 406 may join to form a single face.

Illumination beam 403 enters input face 405 of prism 404, reflects from first angled face 408 and is directed to detection face 407. Light reflecting from detection face 407 further reflects from second angled face 409 and exits prism 404 via output face 406. A camera 410 images a portion of detection face 407. Images captured by camera 410 can be analyzed as described above to detect attachments of nucleotides to targets within flow cell 101. A cover glass 412 may also be present.

The systems of FIGS. 3 and 4 are examples of systems that operate in a reflection mode. In these systems, illumination and detection are performed from the same side of flow cell 101, and light reaches the sensor by reflection from the detection surface of the prism.

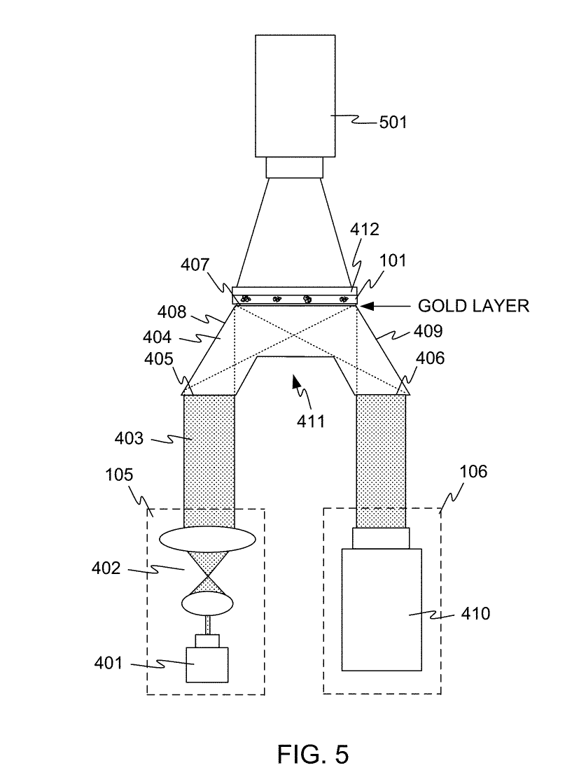

FIG. 5 illustrates a system 500 in accordance with other embodiments of the invention. Some portions of system 500 are duplicated from system 400 shown in FIG. 4, and are given the same reference numbers. In system 500, an additional camera 501 is placed on the opposite side of flow cell 101 from illumination system 105, and images a plane at flow cell 101.

Camera 501 may sense changes within flow cell 101 (and therefore bindings of test nucleotides to the sample) using surface plasmon enhanced fluorescence (SPEF) imaging. An optical bandpass filter may be included in camera 501 to block non-fluorescence wavelength light and only allow fluorescence light to pass through. In SPEF, fluorescence of markers within the sample is excited by the surface plasmon effect. The fluorescence can be detected by camera 501, to detect locations where test nucleotides have attached to the sample. Camera 410 preferably operates in parallel with camera 501. Thus, system 500 operates in both a reflection mode and a transmission mode. Camera 401 performs sensing from the same side of flow cell as illumination system 105 (reflection mode), while camera 501 senses from the opposite side (transmission mode).

In other embodiments, detection surface 407 of prism 404 may not be plated, and camera 501 may perform total internal reflection fluorescence (TIRF) imaging. In TIRF, the fluorescence is excited by an evanescent wave resulting from the total internal reflection of the illumination light from the interior surface of the prism. SPEF can produce images with better signal-to-noise characteristics than TIRF. A system using TIRF is an example of a system operating in a transmission mode, because the sensing is performed from the opposite side of flow cell 101 from illumination system 105.

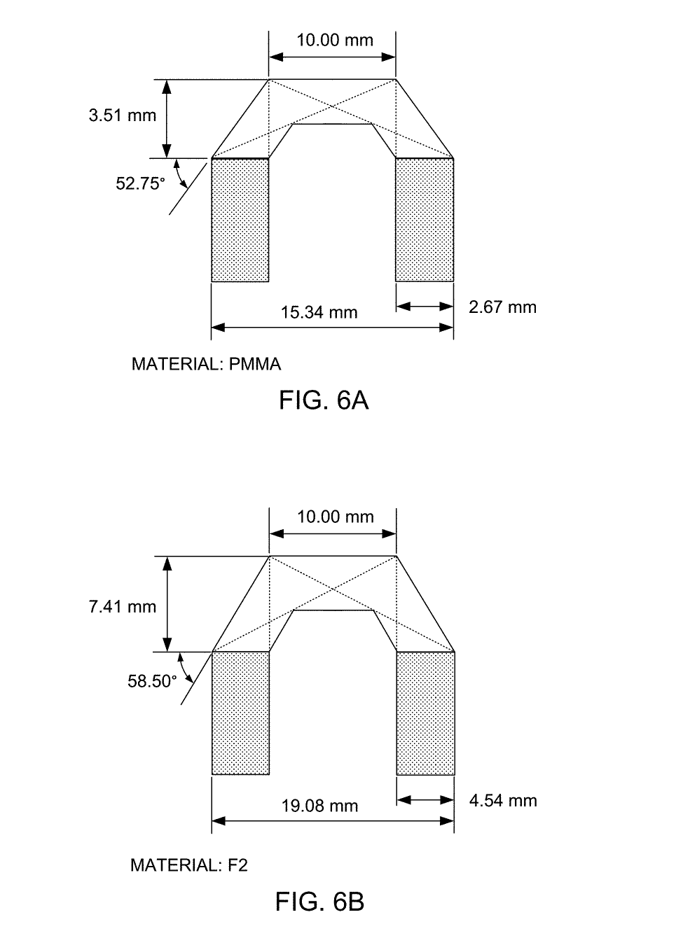

The surface plasmon effect is very sensitive to the configuration of the system, including the materials of the components, the wavelength of light used, and the angle of incidence of light on the surface where it is desired to produce plasmons. The index of refraction of the prism is an important parameter. FIGS. 6A and 6B illustrate two trapezoidal prisms that may be suitable for use in the systems such as those of FIGS. 4 and 5.

In a system such as those shown in FIGS. 3-5, the detection face of the prism may be in direct contact with analytes, for example, being integral to a flow cell through which analyte-containing reagents are delivered. As such the detection face of the prism can be functionalized with a reactive material such as a mixed alkanethiol monolayer to provide the ability to bind avidin, neutravidin, or streptavidin, onto the slide which can then bind biotinylated probes such as amplicons, priming sequences, barcode sequences, or other capture elements. The detection face may be either unpatterned or patterned. Patterning of the sensing element can be achieved by either spotting reagents into an ordered array of spots with a microarraying device, or by selectively depositing reagents to the surface using a flow cell to create sensing regions.

In some systems such as those shown in FIGS. 3-5, modification of the surface properties of the detection surface can provide the ability to present chemical moieties that provide a high level of specificity for sensing. A number of strategies can be adopted depending on the material comprising the sensing element.

For example, in some embodiments, the gold or other thin-film is coated with a self-assembled monolayer (SAM) of alkanethiol molecules. The monolayer is comprised of a mixture of inter polyethylene glycol (PEG) chains and biotin terminated alkanethiol chains. The mixture is tailored to allow optimal spacing between the biotin moieties for binding of avidin, neutravidin, or streptavidin. In other embodiments, the gold thin film can be modified with amine-terminated, carboxy-terminated, or glycidoxypropyl-terminated alkane thiols to allow for derivatization with heterobifunctional cross-linking agents. These surface modifications can also serve as an adhesion layer for physically adsorbed polymers (e.g. proteins, polylysine, dextrans, polydopamine, etc). Other surface chemistries for attaching biomolecules to a surface are well known, and include hydrogels (acrylamide, agarose,), polymers (polylysine, dextran, polydopamine, poly acrylic acid, pHEMA,), bifunctional crosslinkers with reactive endgroups (comprising sulfhydryl, carboxyl, hydroxyl, amino, azido, alkyne, phosphonic acid,). Adhesion layer formed by self assembly, immersion, dip coating, spin coating, electodeposition, electroless deposition, vapor deposition, Langmuir-Blodgett film transfer, reversible addition fragmentation transfer—RAFT, atom transfer radical polymerization—ATRP. Surfaces can be activated/cleaned by plasma, UV ozone, chemical, radiation, or other means. A scattering label may be conjugated to the polymerase molecule and in the presence of the correct base the density of labeled polymerases can create a detectable scattering cross section. The scattering label may be comprised of gold nanoparticles in the size range of 5-100 nm dia. Detection can be accomplished in reflectance or transmission mode

Flow Cell Arrangements

In some embodiments it is desirable that fluids flowing through flow cell 101 exhibit uniformity of velocity and cover the array of features on flow cell 101 as nearly completely as possible.

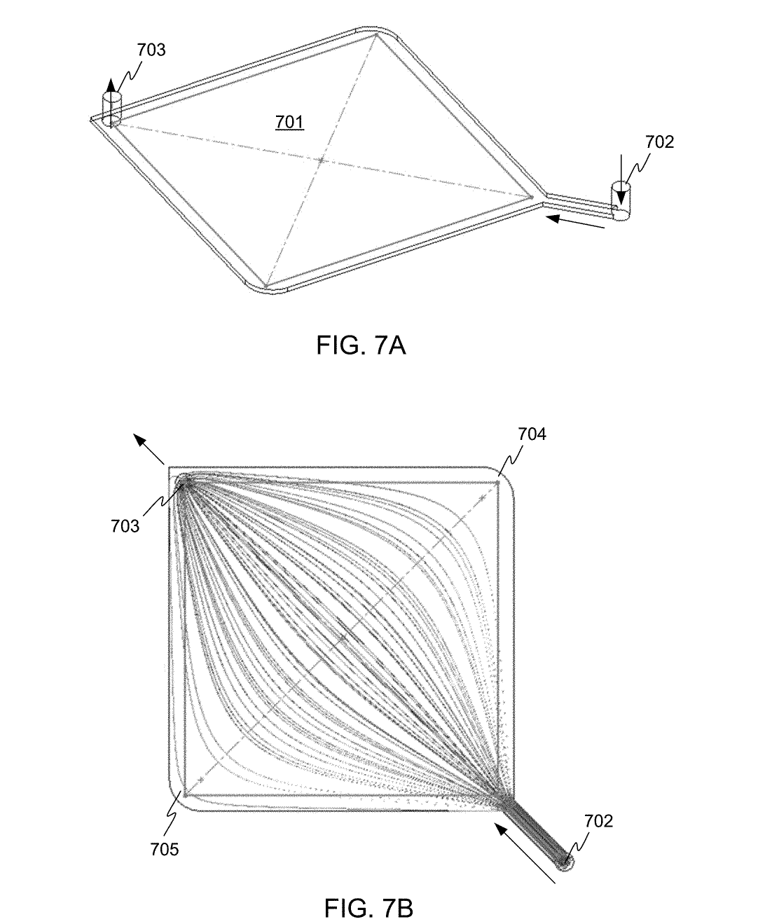

FIG. 7A illustrates a flow cell cavity arrangement in accordance with embodiments of the invention. The cavity illustrated in FIG. 7A may be covered by a transparent structure, and defines a thin, flat recess 701 into which a sample may be placed. An inlet port 702 allows introduction of fluids to the cavity, and an outlet port 703 provides an escape route for the fluids after they have traversed recess 701. In this example arrangement, fluids are introduced to flow cell 101 at one corner of the flow cell and exit at the opposite corner.

FIG. 7B illustrates the flow of fluids through the flow cell arrangement of FIG. 7A. As can be seen, corners 704 and 705 tend not to receive significant fluid flow.

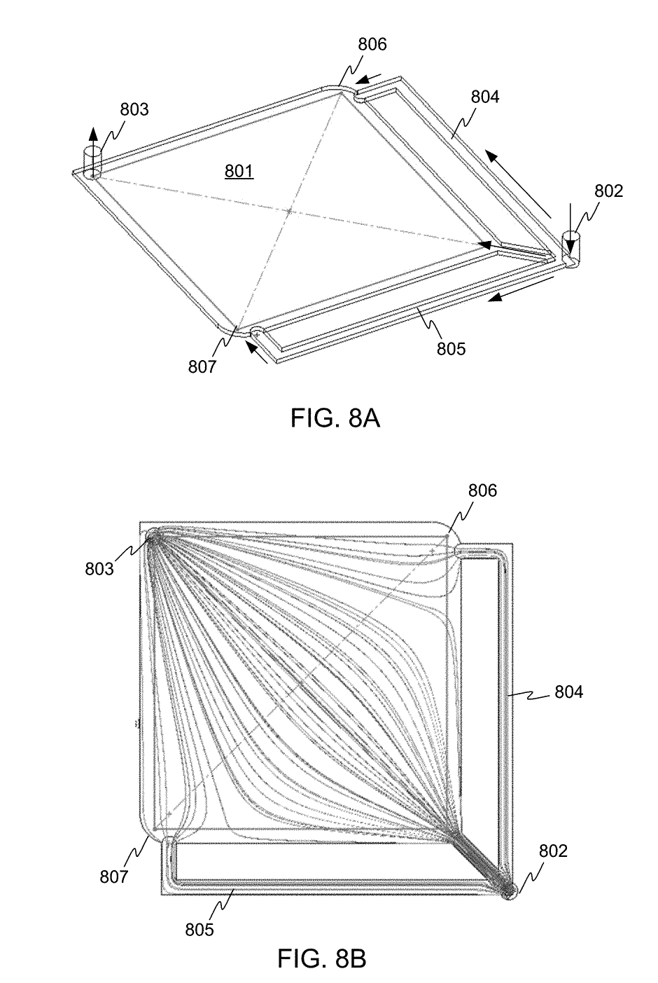

FIG. 8A illustrates a flow cell cavity arrangement in accordance with other embodiments of the invention. The cavity illustrated in FIG. 8A defines a thin, flat recess 801 into which a sample may be placed. An inlet port 802 allows introduction of fluids to the cavity, and an outlet port 803 provides an escape route for the fluids after they have traversed recess 801. In addition, channels 804 and 805 direct fluid from inlet port 802 to locations near corners 806 and 807, in addition to fluid flowing into the corner nearest inlet port 802.

FIG. 8B illustrates the flow of fluids through the flow cell arrangement of FIG. 8A. As can be seen, corners 806 and 807 tend to receive somewhat more fluid flow than in the cavity arrangement of FIG. 7A.

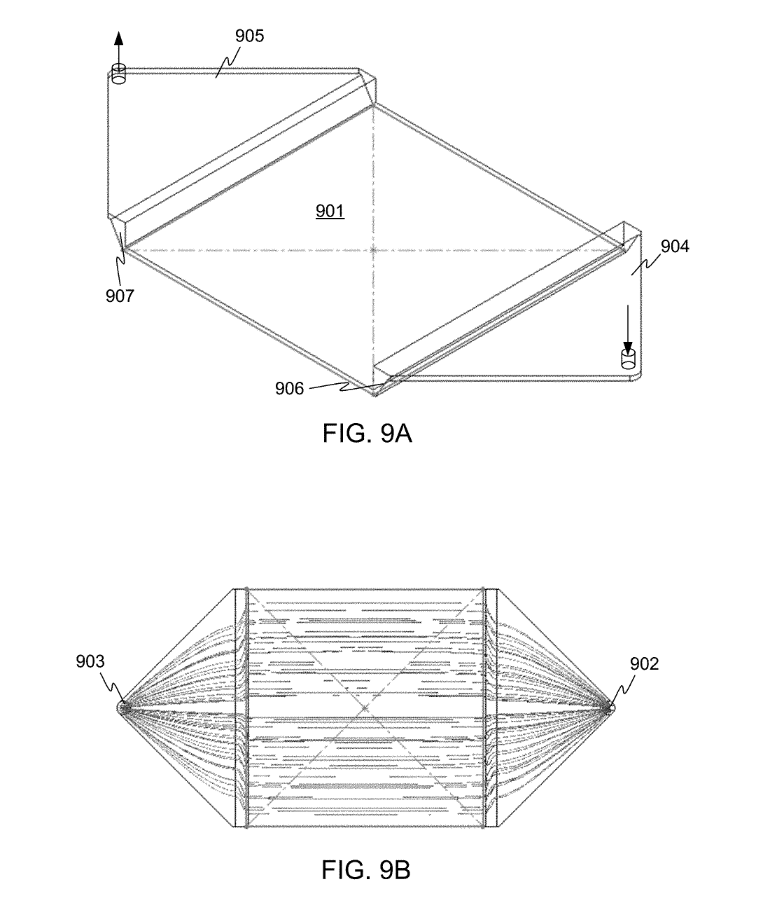

FIG. 9A illustrates a flow cell cavity arrangement in accordance with other embodiments of the invention. The cavity illustrated in FIG. 9A defines a thin, flat recess 901 into which a sample may be placed. An inlet port 902 allows introduction of fluids to the cavity, and an outlet port 903 provides an escape route for the fluids after they have traversed recess 901. In addition, inlet port 902 is at a corner of a triangular lead in channel 904 that joins recess 901 at one edge, and carries fluids from inlet port 902 to recess 901. Similarly, a triangular lead out channel 905 joins recess 901 at one edge, and carries fluids from recess 901 to outlet port 903 at the triangle corner apart from recess 901. Preferably, recess 901 is displaced vertically from lead in and let out channels 904 and 905, so that the fluids undergo a vertical shift 906 during incoming flow and an opposite vertical shift 907 upon leaving recess 901.

FIG. 9B illustrates the flow of fluids through the flow cell arrangement of FIG. 9A. Lead in channel 904 and lead out channel 905 may have only straight edges, may have only curved edges, or may have a combination of straight and curved edges. The lead in and lead out channels may have constant or varying cross sections.

The fluidic channel carrying fluids from reservoirs 102 to flow cell 101 may comprise a simple channel, or may contain more complex structures to enable mixing, sorting, switching, or perform other fluidic operations. The order of fluids entering the channel are controlled by valves 103. Valves 103 may comprise a thin silicone layer over an inlet port. Each valve is normally closed by using either pneumatic or mechanical force. When closed, the silicone material is pressed over the opening of the inlet port with sufficient force to stop the flow of reagent through the inlet. Additional reagents may flow around the valve due to bypass channels that go around the occluded valve.

The fluidic channel may be contained in a disposable manifold piece that connects to reagent containing vessels. The reagents may be contained within sealed vessels with tubing connecting the vessels to the fluidic channel, or may be contained in a separate disposable reagent pack that attaches to the device manifold.

The fluids may be driven either by pneumatic pressure or by mechanical force, for example by a pump such as pump 104 shown in FIG. 1. In a preferred embodiment, the reagent vessels are connected to either an external pressurized gas source, or an internally mounted pneumatic pump. Pneumatic pressures utilized may range from 0 psi to 40 psi. Optionally, fluidics can be simplified by implementing a sipper/dispenser configuration where a syringe on an XYZ stage aspirates and dispenses reagents onto and away from the flow cell. Capillary forces can also be used to move liquid in and out of the flow cell.

Instrument Designs

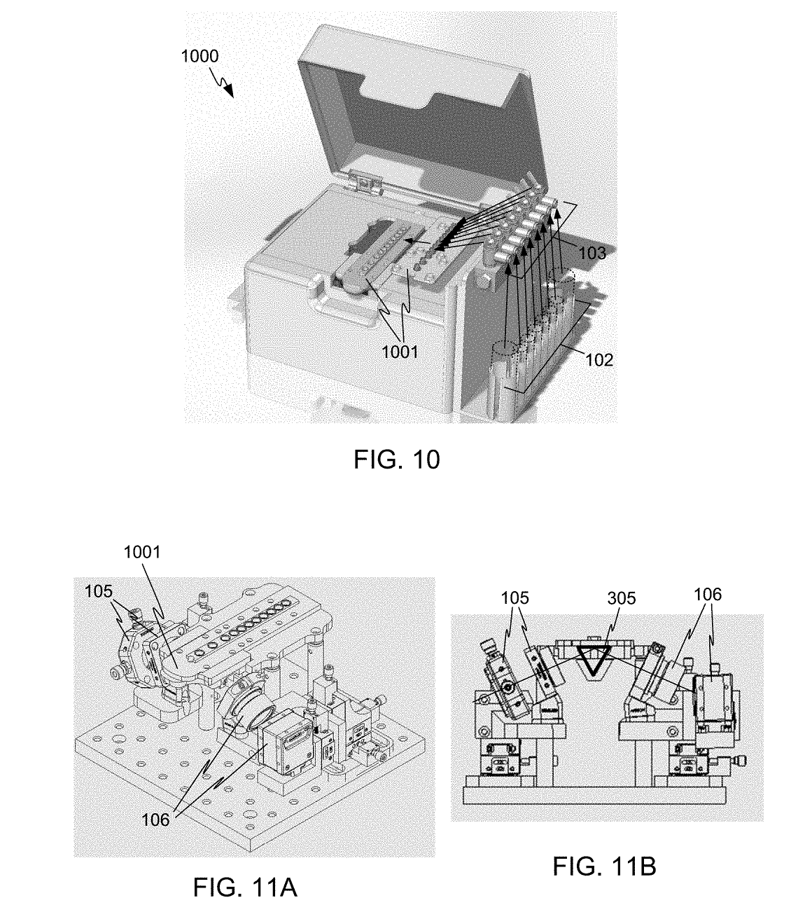

FIG. 10 illustrates an instrument 1000 in accordance with embodiments of the invention. Instrument 1000 includes a flow cell, an illumination system, and a sensing system, for example of the kinds described above. In some embodiments, instrument 1000 is a self-contained unit including all necessary subsystems to perform a sequencing reaction, and includes an illumination system, a detection systems, a fluidic module, a disposable reagent pack, electronics for data acquisition and control, and software for data acquisition and control. Additionally, the instrument can include all necessary subsystems to create nucleic acid features on the surface of a flow cell. Detection of the sequencing reaction may be achieved by label-free optical detection enabled by surface plasmon resonance sensing (SPR), or surface plasmon resonance imaging (SPRi), or other methods. It will be understood that the instrument can be similarly designed for other detection purposes in addition to, or as an alternative to, nucleic acid sequencing. Those skilled in the art will be able to modify the design exemplified below in view of the desired detection purpose.

Instrument 1000 includes reservoirs 102 for holding the various reagents used in operation of the instrument, and valves 103. Fluids containing the reagents are taken from reservoirs 102 under control of valves 103 in the correct sequence and amounts, and supplied to a disposable fluidic interface 1001 (shown partially disassembled in FIG. 10 and described in more detail below). The fluid flows are shown schematically in FIG. 10. In practice instrument 1000 preferably includes tubing, hoses, or similar conduits for carrying the fluid flows.

FIGS. 11A and 11B illustrate instrument 1000 with its cover removed, so that certain interior components are visible. Illumination system 105 directs light to prism 305, where it reflects toward sensing system 106 after being affected by the reactions occurring in flow cell 101 adjacent the top surface of prism 305. Illumination system 105 may include a light emitting diode, a laser, or another kind of light source.

The angle of the incident light with respect to the surface of the sensing can be varied to measure changes in reflected intensity, or remain fixed. Preferably, the angle of the incident light is held fixed and changes in reflected intensity are measured as the sequencing reaction proceeds. The instrument can be configured in such a way that either increasing or decreasing intensity can correspond to the detection of a sequencing step.

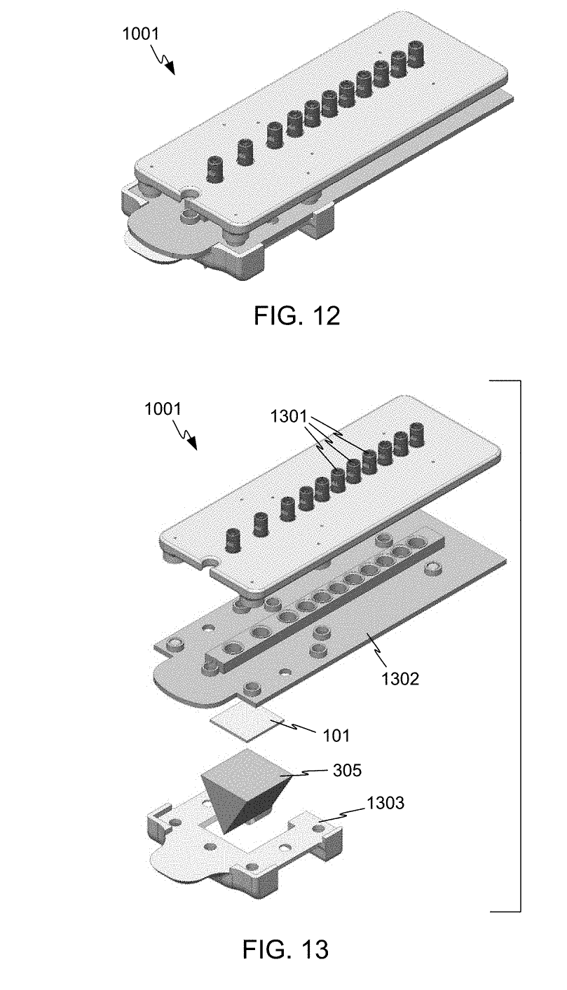

FIG. 12 illustrates disposable fluidic interface 1001 of instrument 1000 in isolation, and FIG. 13 is an exploded view of disposable fluidic interface 1001. Disposable fluidic interface 1001 includes fluidic connection ports 1301, valve cartridge 1302, flow cell 101, prism 305, and a prism mount 1303.

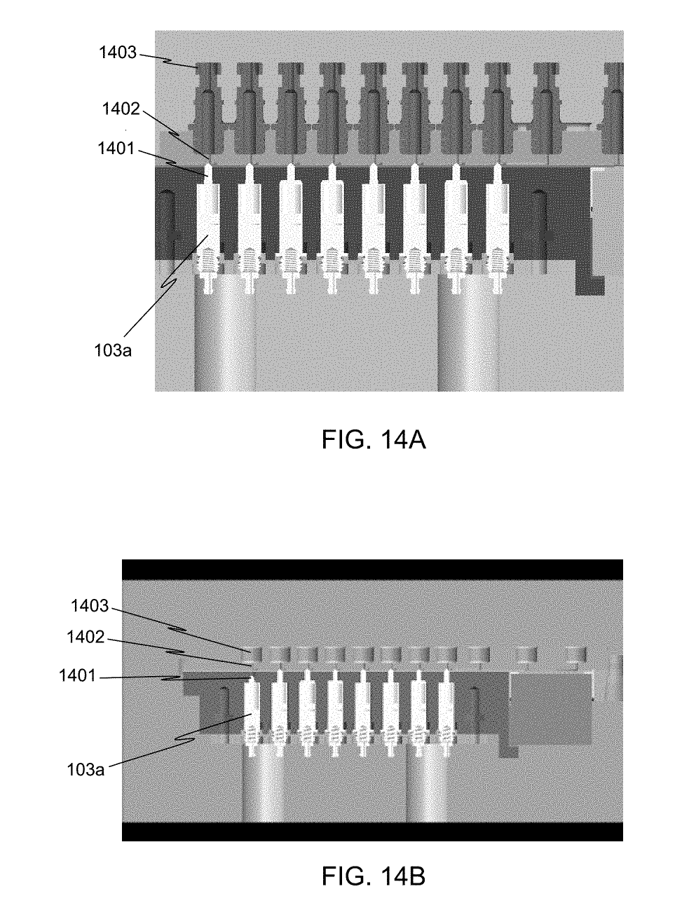

FIG. 14A illustrates a cutaway view of disposable fluidic interface 1001, showing valves 103 in their normally closed positions. For example, a plunger 1401 of valve 103a is in a raised position, closing off channel 1402 so that no fluid flows from port 1403. FIG. 14B shows valve 103a in its open position. Plunger 1401 is now in a lowered position, unblocking channel 1402, and allowing the flow of fluid from port 1403.

Power and control signals for the various components of instrument 1000 are controlled utilizing an internal breakout board, or other data acquisition (DAQ) and control device. In a preferred embodiment, a custom breakout board providing a unified interface for all subsystems is connected to an external power source or a battery, a DAQ card, a light source, and pressure regulation device. This breakout board may be connected to a computer by a USB 3.0 cable or another kind of interface. Signals from the computer are routed through the breakout board to control subsystems. The sensing system may be a camera, which may be connected directly through the computer or via the breakout board. The instrument may be controlled via custom written DAQ software. The software allows for control of all subsystems of the instrument, collection and saving of data (e.g. text files, image files, etc.) as well as real-time analysis of the collected data (e.g. data manipulation, base calling, etc.). Instrument 1000 may be especially suited for low-throughput sequencing of 1-1000 amplicons for targeted gene panels. In some embodiments, control may be accomplished using low cost off-the-shelf control components such as the Raspberry Pi computer developed by the Raspberry Pi Foundation or simple controllers available from Phidgets, Inc. of Calgary, Alberta, Canada.

Cartridge

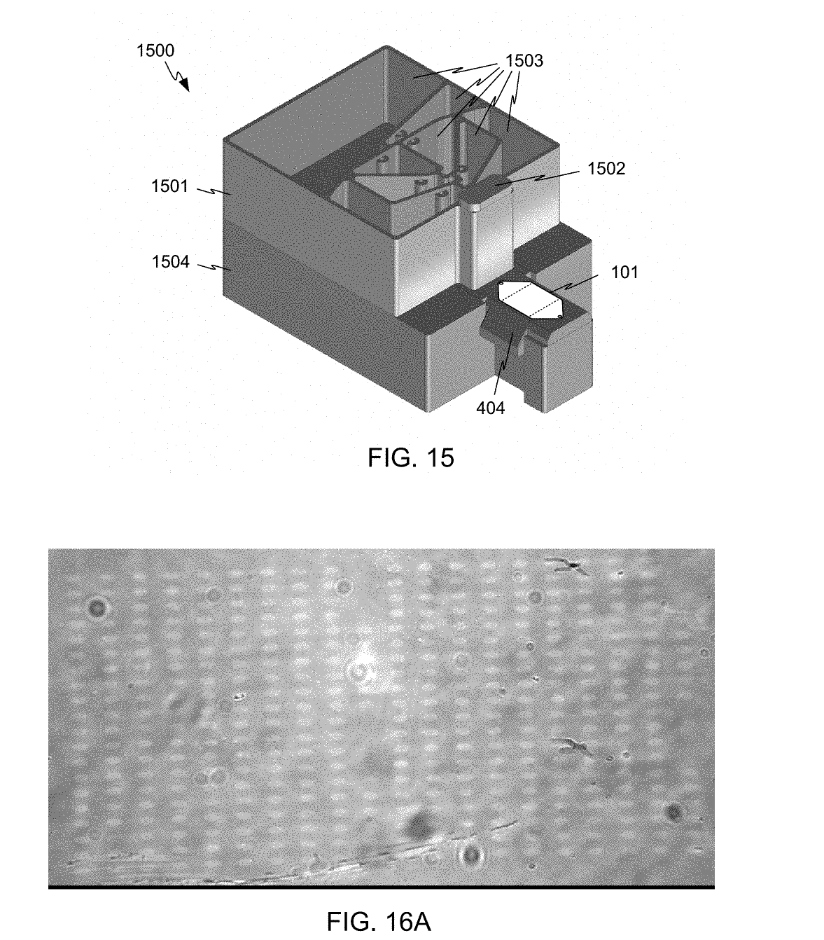

FIG. 15 illustrates a cartridge 1500 according to embodiments of the invention. Cartridge 1500 incorporates several components of an instrument such as instrument 1000 into a self-contained disposable unit. Cartridge 1500 is shown in FIG. 15 with its top cover removed, so that internal details are visible. Cartridge 1500 includes a housing 1501 defining a sample well 1502 and a number of reagent wells 1503, holding the sample and various reagents needed for a sequencing operation. A prism such as prism 404 is included, and a flow cell 101 resides adjacent the prism. Alternatively, the prism and flow cell can be integrally formed such that reagents in the flow cell are in direct contact with a facing surface of the prism.

A set of valves is also included (but not visible in FIG. 15), for controlling flow of the various sample and reagent fluids to flow cell 101. The valves may be, for example, fluidic valves, made from a thin silicone layer over an inlet port. Such a valve is normally closed by using either pneumatic or mechanical force. When closed the silicone material is pressed over the opening of the inlet port with sufficient force to stop the flow of reagent through the inlet. Additional reagents may flow around the valve due to bypass channels that go around the occluded valve. Fluid from any particular one of the reservoirs can be individually supplied to the flow cell by opening of the respective valve of the particular reservoir and closing the other valves.

Preferably, cartridge 1500 is disposable after being used for one sequencing task. The disposability may be facilitated by designing the components of cartridge 1500 for low cost. For example, housing 1501 may be configured such that it can be fabricated by injection molding, and prism 404 may also be fabricated from an injection molded polymer.

Cartridge 1500 also includes one or more waste reservoir wells 1504, for receiving sample and reagent fluids after they have passed through flow cell 101. For ease of use and biocontainment, the cartridge could be self-contained such that all sample and reagents are kept on the cartridge. The cartridge could be aseptically sealed to prevent contamination.

Surface Modification Strategies

Modification of the surface properties of the detection surface of a prism such as prism 305 or prism 404 can provide the ability to present chemical moieties that provide a high level of specificity for sensing. A number of strategies can be adopted depending on the material comprising the sensing element.

For example, in some embodiments, a gold thin-film is coated with a self-assembled monolayer (SAM) of alkanethiol molecules. The monolayer is comprised of a mixture of inter polyethylene glycol (PEG) chains and biotin terminated alkanethiol chains. The mixture is tailored to allow optimal spacing between the biotin moieties for binding of avidin, neutravidin, or streptavidin.

Alternately, the gold thin film may be modified with amine-terminated, carboxy-terminated, or glycidoxypropyl-terminated alkane thiols to allow for derivatization with heterobifunctional cross-linking agents. These surface modifications can also serve as an adhesion layer for physically adsorbed polymers (e.g. nucleic acids, proteins, polylysine, dextrans, polydopamine, etc.).

Array Barcoding Example—Microspotted Array

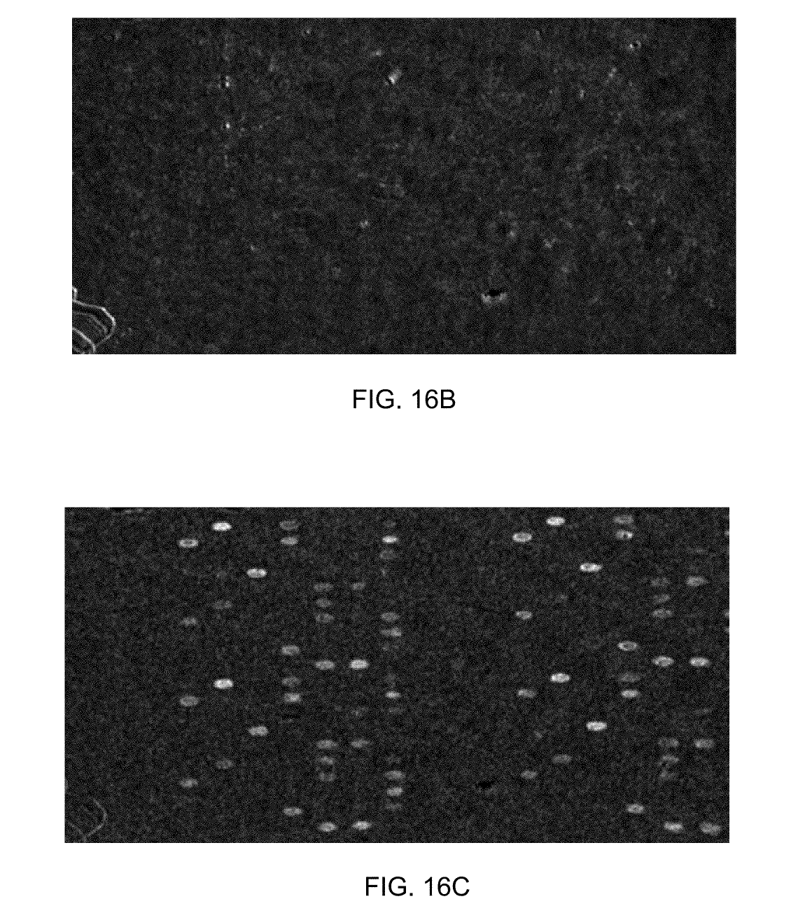

FIG. 16A shows an unprocessed surface plasmon resonance (SPR) image of a microspotted array on a gold thin film. An image such as FIG. 16A may be obtained, for example, using a system such as is shown in FIGS. 3-5. A microspotted array includes a microarray of DNA spots created on the gold thin-film substrate by microspotting of reagents into an ordered array, with defined spot sizes and lattice spacing.

In one embodiment, a drop containing 50 μg/ml of streptavidin is first placed on an alkanethiol-modified gold thin-film. The spot is allowed to incubate under 70% humidity for 2 hours. The same spot is then incubated with a drop of biotinylated-DNA containing solution. The DNA can comprise either a piece of template DNA, a primer sequence, or a universal bar code sequence for capturing PCR products, or other types of biomolecules. The resulting spotted arrays are imaged using SPRi, for example to obtain an image such as FIG. 16A. This strategy enables performing sequencing reactions on multiple templates strands simultaneously.

FIG. 16B illustrates a processed SPR image with background subtraction prior to exposure to sequencing reagents. FIG. 16C shows the change in relative reflected intensity on the microspotted chip after exposure to sequencing reagents.

Array Barcoding Example—Flow Patterned Array

In some embodiments, the sensing chip can be patterned using a microfabricated flow cell to directly expose the desired regions of the chip to different templates. In one example, a PDMS flow cell was fabricated using soft lithographic method. The flow cell was then placed on top of the sensing chip, and reagents were driven using a syringe to create a vacuum at the outlet. A first a solution containing 10 μg/ml of streptavidin in KCl buffer was flowed over the chip and allowed to incubate for 15 minutes. The streptavidin containing solution was then washed out with buffer. This was then followed by a biotin-DNA containing solution in the same buffer and incubated for 25 minutes. The biotin-DNA containing DNA solution was then washed out with the buffer. The chip was immediately transferred to the instrument for sequencing.

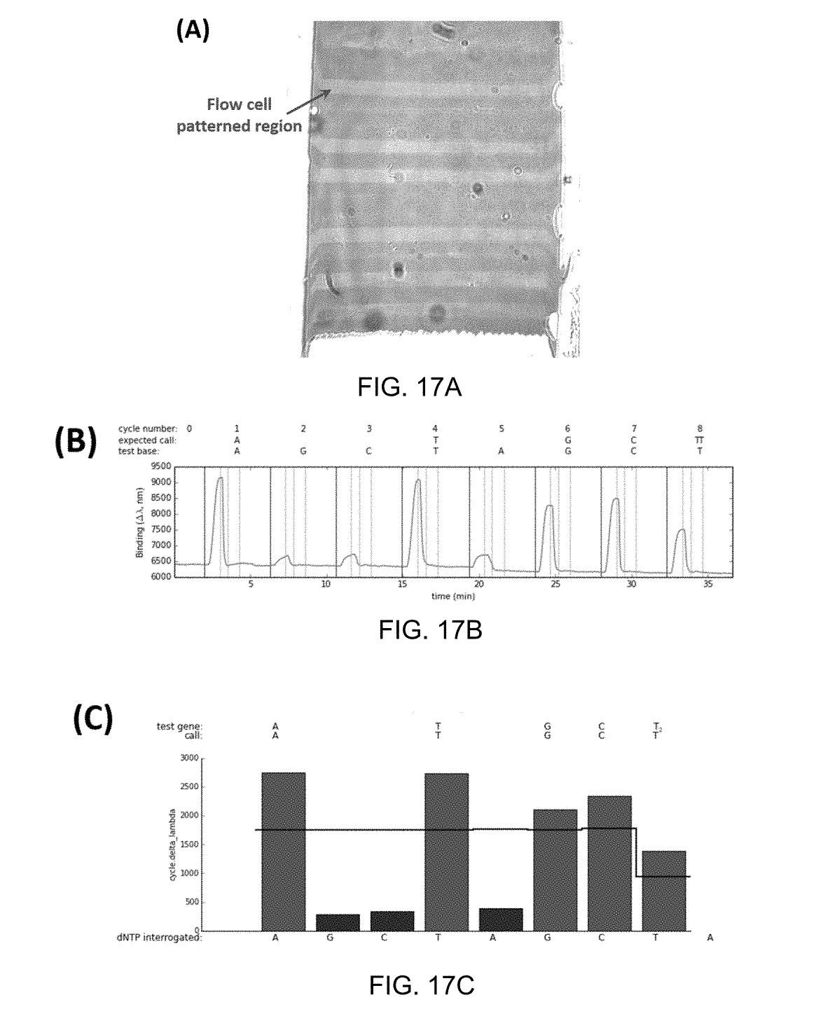

During sequencing, the DNA patterned regions were exposed to sequencing reagents. FIG. 17A shows an SPR image of the flow patterned chip. Streptavidin/DNA containing regions appear as regions of higher reflectance (brighter) compare to surrounding regions. FIG. 17B shows raw sequencing data collected from a region contain a region of the phiX bacteriophage genome. FIG. 17C shows the resulting positive (5 taller bars) and negative (3 shorter bars) base calls from the raw data showing successful sequencing.

Nanohole Array Sensing

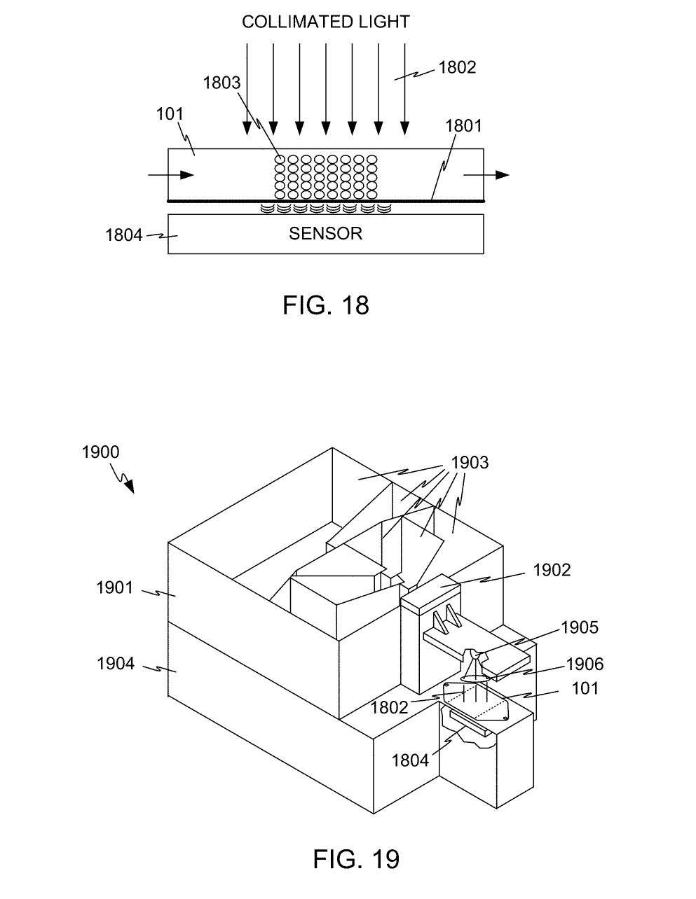

In some embodiments, sensing technologies other than those described above may be used, for example nanohole array sensing. FIG. 18 schematically illustrates nanohole sensing. A flow cell 101 is in close proximity or contact with a metallized surface 1801 having a pattern of nanoholes formed through the metal layer. The metal layer may be made of gold, silver, aluminum, or another suitable metal. Flow cell 101 is illuminated by incoming collimated light 1802. Targets having bound proteins 1803 are near or attached to metallized surface 1801.

The size and spacing of the nanoholes may be selected in accordance with the wavelength of light being used in the system, but in one embodiment, the nanoholes may each be about 200 nm in diameter and the nanoholes may be arranged in a grid having rows and columns spaced about 450-475 nm apart. This example arrangement may be suitable for use with light having a wavelength of 650 nm, but other wavelengths, spacings, or both may be used. The nanoholes need not be on a rectangular grid.

In any event, the diameter of the nanoholes is smaller than the light wavelength. In a phenomenon known as extraordinary optical transmission (EOT), some of incoming light 1802 is transmitted through metallized layer 1801 and reaches array light sensor 1804. Array light sensor 1804 may be a charge coupled device (CCD) sensor, a complementary metal oxide semiconductor (CMOS) sensor, or another kind of sensor. The intensity of light 1805 reaching sensor 1804 is affected by the binding of proteins 1803, so that comparison of ‘before’ and ‘after’ images taken by sensor 1804 can reveal sites where proteins have bound. Specifically the intensity of the light is very sensitive to the bulk and surface refractive indexes of the materials at and near surface 1801. By sequentially flowing test reagents through flow cell and analyzing images from sensor 1804, sequencing can be performed as described above.

The system of FIG. 18 may perform “lensless” or “contact” imaging. Because sensor 1804 is in very close proximity to surface 1801, the effect of diffraction is minimized, and image quality can be maintained.

With a well-collimated beam, the sensing resolution is limited by the diffraction from the microarray spots. The angle of diffraction is determined from sin Θ=λ/dₛₚₒₜ. Thus, the additional blur diameter due to diffraction in a “contact” imager is given by d_{diff}=L. tan(arcsin(λ/dₛₚₒₜ)), where L is the distance from the microarray and the sensor surface, λ is the wavelength of light being used, and dₛₚₒₜ is the diameter of the features on the microarray causing diffraction. Thus, the blur diameter is smaller with smaller L. Some example dimensions and their performance are given in Table 1 below:

TABLE 1
Performance of lensless contact imaging.

| L (mm) | dₛₚₒₜ (μm) | d_{diff} (μm) | |
|---|---|---|---|
| 1 | 30 | 22 | Resolvable |
| 1 | 100 | 6.5 | Well-resolvable |
| 10 | 50 | 130 | Unresolvable |
| 10 | 100 | 65 | Resolvable |

Referring again to FIG. 18, collimated light 1802 may be generated by any suitable means, for example using a light emitting diode (LED) with a condenser lens, using a laser with a beam expander, or by other methods. In some embodiments designed for low cost, collimated light may be derived from ambient light. In some embodiments, polarizers may be present, for example one above flow cell 101 and one between surface 1801 and sensor 1804. (The polarizers are not shown in FIG. 18). The polarizers may preferably be oriented with their polarization directions orthogonal, so that they perform cross polarization.

In some embodiments, the components of FIG. 18 may be incorporated into a disposable module. For example, FIG. 19 illustrates a module 1900 including the sensing system of FIG. 18. Similar to cartridge 1500 shown in FIG. 15, cartridge 1900 includes a housing 1901 defining a sample well 1902 and a number of reagent wells 1903, holding the sample and various reagents needed for a sequencing operation. Cartridge 1900 may also include a waste reservoir 1904.

Cartridge 1900 also includes a light source 1905 and a collimating lens 1906, for generating collimated light 1802. Some of light 1802 reaches array light sensor 1804 after being affected by reagents within flow cell 101 and passing through a nanohole array (not visible in FIG. 19), for imaging as is described above. Various fluidic and electrical connections are not shown in FIG. 19 for clarity. Other kinds of light sources may also be used, as described above.

In some embodiments, cartridge 1900 may be disposable, for example, being discarded after a single sequencing use.

Nanohole Sensing Example

In an example of nanohole sensing, a nanohole array (NHA) was coated with a lysine-fixable, biotinylated dextran (Life Technologies, D-1956). The dextran was resuspended in deionized water at a concentration of 1 mg/ml. A droplet of the dextran solution was then placed on the NHA and allowed to dry. The NHA was cut to a square of approximately 4×4 mm and fixed to a 1″ diameter, circular glass slide using double-sided sticky tape.

A fluidic cell was fabricated by cutting a channel into a 3 mm thick, 1″ diameter piece of PDMS. The fluidic channel was then placed over the NHA, making sure the PDMS was well, but reversibly, adhered to the glass slide. The fluidic cell was then brought into contact with a custom fabricated lid with inlet and outlet ports for flowing reagents. Pressure was applied to the two pieces to create a fluid tight seal.

The NHA was illuminated using a 15 mW laser diode with a nominal emission wavelength of 670 nm. Light transmitted through the NHA was imaged using a Grasshopper 3 (Point Grey, Richmond, Canada). Image acquisition was performed using a custom routine written in Labview VI. Image analysis and intensity measurements were performed using Image J.

Prior to measurement, the fluidic cell was primed with 1×PBS to ensure all air bubbles were removed and the biotin-dextran coating was rehydrated. A solution containing 50 μg/ml of Streptavidin in 1×PBS was injected into the flow cell. Binding of the resulting streptavidin layer was monitored by measuring the change in light transmission through the NHA. Streptavidin was allowed to bind to the biotin-dextran layer for approximately 100 seconds, followed by washing with excess 1×PBS.

A 100 nM solution of biotinylated template DNA was prepared with a suitable primer sequence in a solution with a final concentration of 2×PBS. Prior to introduction of the primer/template DNA, 2×PBS solution was washed through the flow cell to minimize the change in bulk dielectric due to ionic strength of the solution.

Primer/template DNA was then injected into the flow cell and allowed to bind to the streptavidin layer for approximately 100 seconds. The primer/template DNA solution was washed out with excess 2×PBS. After wash with 2×PBS, the flow cell was washed with 1×PBS to prepare for the subsequent DNA polymerase solution.

Four milliliters of a 100 nM solution of the Klenow fragment of DNA polymerase I was prepared in a 1× solution of taq buffer with 100 nM dATP, which corresponded to the conjugate nucleotide of the next correct base of the template sequence. 4 μl of 1M SrCl₂ was added to the solution to stabilize the polymerase/DNA/nucleotide complex without incorporating the nucleotide into the growing strand. The polymerase containing solution was injected into the flow cell, and allowed to incubate for approximately 100 seconds. Polymerase binding was detected by monitoring intensity changes in light transmitted through the NHA. After the association phase, the polymerase solution was washed out with excess 1×PBS.

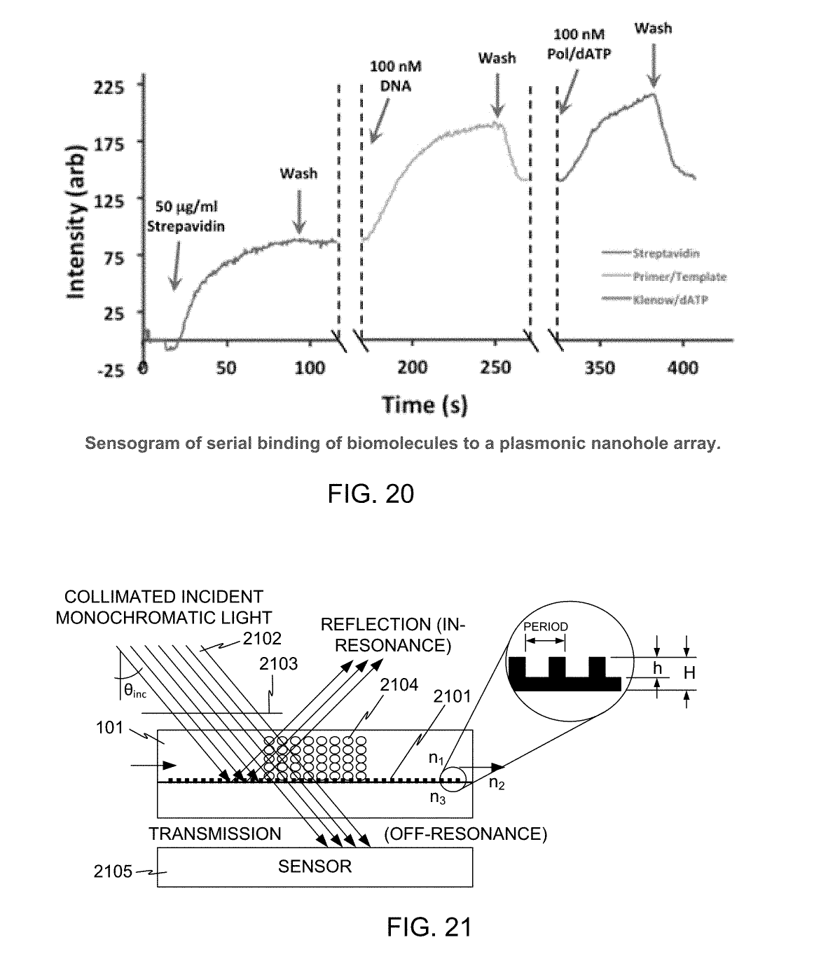

A NHA imaging system was used to detect DNA polymerase binding to a primed strand of template DNA in the presence of dATP. FIG. 20 shows the sensogram recorded. The first binding step involved introduction of a 50 μg/ml solution of streptavidin in PBS into the flow cell. The resulting association of the streptavidin with biotin groups on the dextran/biotin coated NHA was detected by a change in transmitted light intensity. Due to the high affinity of streptavidin for biotin, no significant change in signal was measured after wash with 1×PBS indicating strong binding of the streptavidin layer. Subsequently, biotinylated-DNA hybridized with an appropriate primer sequence was introduced into the flow cell. The primer/template construct association was detected as previously described. Upon wash with 2×PBS the measured signal decreased until a new baseline higher than initial baseline was achieved. Finally, a solution of 100 nM DNA polymerase with 100 nM dATP and SrCl₂ was introduced into the flow cell. The first nucleotide to be recognized on the template strand was thymine, thus the next base to be added to the conjugate strand is adenine. Thus, under these conditions, in the presence of the next correct base the DNA polymerase will bind to the primer/template complex. The binding was observed as a change in transmitted light intensity through the NHA. Upon wash with 1×PBS, the signal returned to the initial baseline indicating that the polymerase/dATP complex dissociated as expected.

Additional information about nanohole array sensing may be found in C. Escobedo et al., “Integrated nanohole Array Surface Plasmon Resonance Sensing Device Using a Dual-Wavelength Source,” Journal of Micromechanics and Microengineering 21, No. 11 (Nov. 1, 2011): 115001, which is hereby incorporated by reference herein in its entirety.

Use of Gratings for Sensing

FIG. 21 illustrates another sensing modality usable in embodiments of the invention. In the example of FIG. 21, a flow cell 101 is placed in proximity to a resonant structure including a grating 2101. Collimated light 2102 may be polarized by a polarizer 2103, and is directed toward flow cell 101. Within flow cell 101, are bound proteins 2104. Changing the period of the grating or angle of incidence (θ_{inc}) can bring a narrow spectral resonance line to match the wavelength of the source light 2102. In addition, reducing the step height (h) of the grating may narrow the resonant peak and increase sensitivity to the surface binding (with some possible compromise in the spatial resolution). An additional polarizer (not shown) may be present between flow cell 101 and sensor 2105. A crossed polarizer transmission configuration allows observation of modes coupled into slab a waveguide. This configuration provides near-zero transmission away from resonant conditions and thus provide better SNR (ratio of the modes coupled to the Photonic crystal to the directly transmitted light). Preferably, the angle of incidence (θ_{inc}) is adjustable, so that the correct incident angle can be set for various combinations of grid period and the indices of refraction of the materials present.

The system of FIG. 21 may also be called a grating-waveguide resonance (GWR) sensing system.

As with nanohole sensing, the intensity of the light reaching sensor 2105 is affected by the binding of proteins 2104, so that ‘before’ and ‘after’ images taken by sensor 2105 can reveal sites where proteins have bound. By sequentially flowing test reagents through flow cell and analyzing images from sensor 2105, sequencing can be performed as described above. The system of FIG. 21 may also perform “lensless” or “contact” imaging, due to the close proximity of grating 2101 to sensor 2105.

Other advantages of the system of FIG. 21 may include that the system is highly tunable to operate at any particular wavelength from UV to IR, and that the sensing grating may be robust in comparison with a thin gold layer.

Additional information about sensing using gratings may be found in Block et al., Optimizing the Spatial Resolution of Photonic Crystal Label-Free Imaging,” Applied Optics 48 No. 34 (Dec. 1, 2009), which is hereby incorporated by reference herein in its entirety.

Grating-Waveguide Resonance Examples

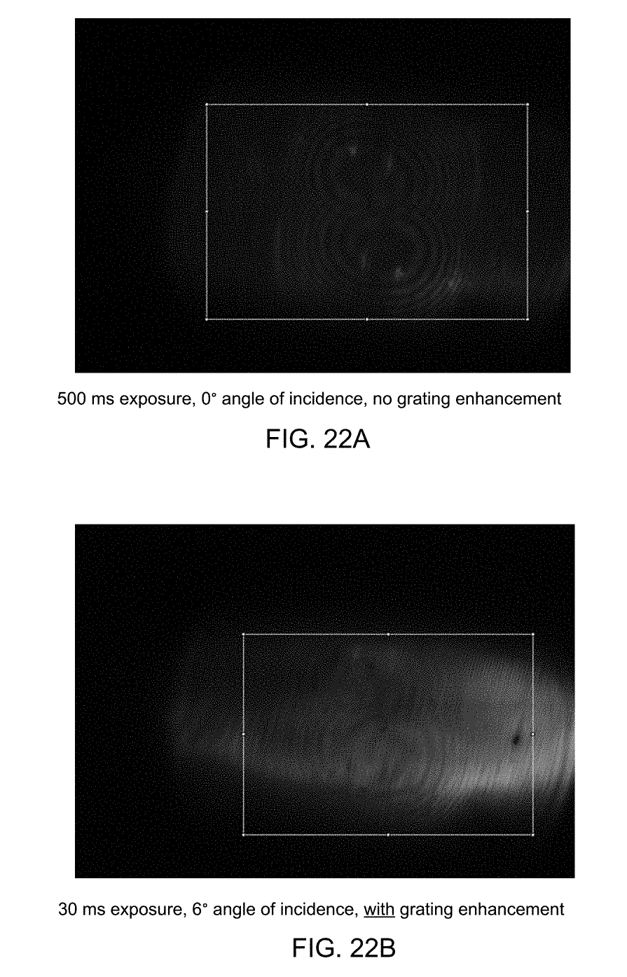

FIGS. 22A and 22B illustrate the effect of grating-waveguide resonance. A UV-ozone cleaned 385 nm pitch grating sample was used, with Alexa-647 labeled bovine serum albumin (BSA) (diluted at 100 μg/ml). About 10-20 μl of fluorophore solution was deposited between a slide and cover glass and illuminated by a 650 nm laser with polarization parallel to the grating lines and axis of sample rotation.

FIG. 22A is an image taken without grating enhancement, and FIG. 22B is an image taken with grating enhancement. As is apparent, much more signal is detected with the grating enhancement, despite the much shorter exposure time. Without enhancement, the mean intensity in FIG. 22A was measured to be about 18.64 (in arbitrary units), while the mean intensity in FIG. 22B was measured to be about 43.67 units. Accounting for the shorter exposure time, the enhancement to the fluorescent intensity was therefore >30λ.

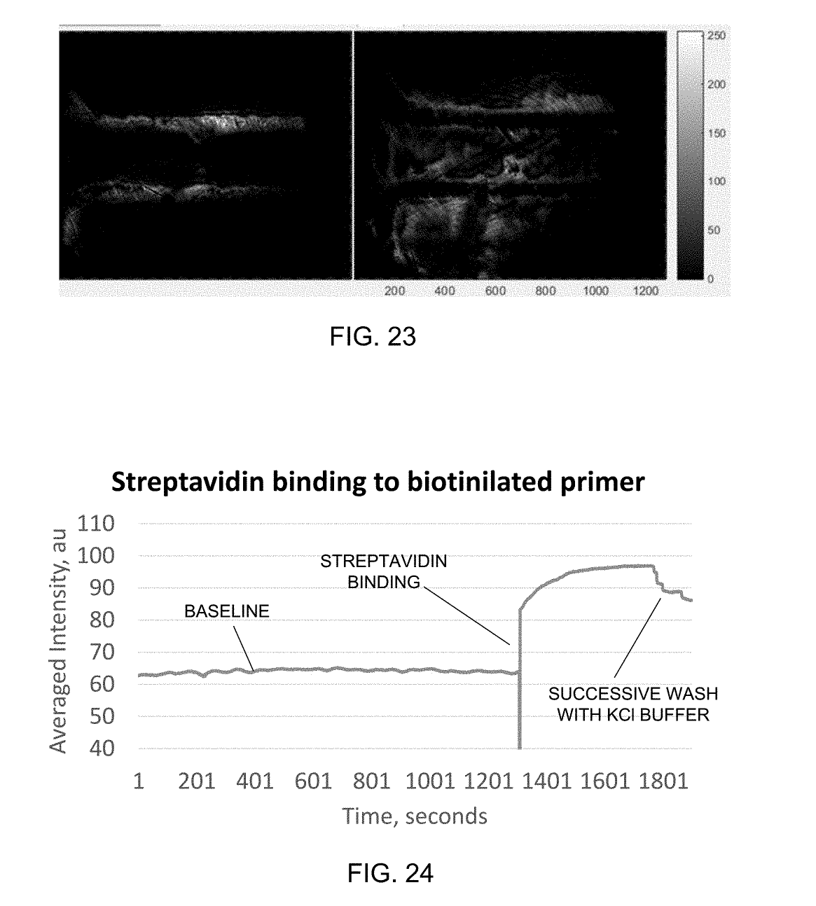

FIG. 23 illustrates images of a flow-patterned substrate taken using grating-waveguide resonance (GWR). The surface imaged is a biotinylated dextran surface chemistry on a UV-ozone cleaned TiO₂ surface. The two images were taken at angles where dextran and bare TiO₂ (respectively) have resonant conditions.

FIG. 24 illustrates averaged intensity readings taken from a polydopamine (universal) surface chemistry functionalization, showing the change in intensity readings upon streptavidin-biotin binding and a subsequent KCl wash.

Gratings Used as Fluorescence-Enhancing Substrates

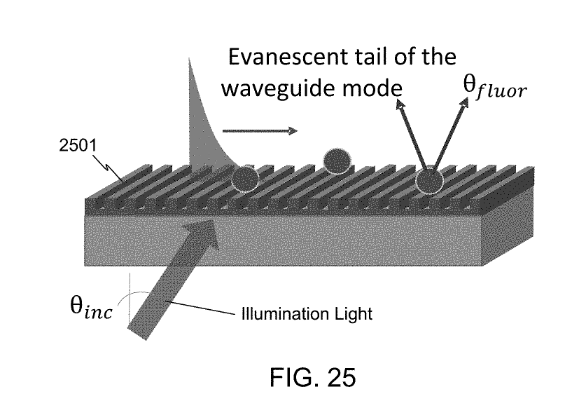

In some embodiments, a grating may be used in a reflection mode, in order to enhance fluorescence. FIG. 25 shows a grating 2501 being used in this manner. Illumination light is supplied to the grating at an incident angle θ_{inc}. The evanescent tail of the waveguide mode excites fluorescence in fluorophores near grating 2501. The fluorescence can then be detected by standard imaging techniques.

At resonance, the incident light is efficiently coupled to the waveguide mode and propagates along the surface thus increasing interaction_with fluorophore molecules, this results in up to a 10× enhancement in excitation efficiency. Additionally, fluorophore molecules are resonantly coupled to the same dielectric waveguide and the resonance angle is θ_{fluor}<θ_{inc} (fluorescence directed to small range of angles rather than 4π). This also improves the collection efficiency and allows using lower NA (cheaper) objectives. Furthermore due to the Purcell effect spontaneous emission rate is higher. Both effects lead to an additional >20× enhancement in emission and collection efficiency of fluorescence radiation. The total fluorescence increase is the product of these factors, and may produce >300× enhancement compared to the fluorescence on a planar substrate.

Additional information about the use of gratings to excite fluorescence may be found in Block et al., Optimizing the Spatial Resolution of Photonic Crystal Label-Free Imaging,” Applied Optics 48 No. 34 (Dec. 1, 2009), previously incorporated by reference.

Surface Plasmon Enhanced Fluorescence Example

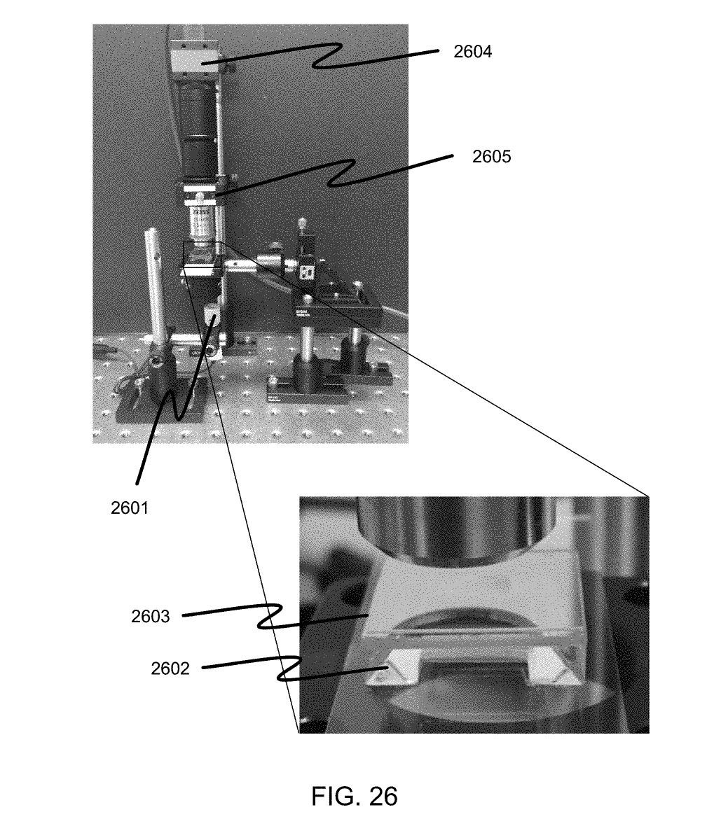

In an experimental run, nanoballs similar to nanoballs 201 were generated using the technique of rolling circle amplification (RCA). A test system as shown in FIG. 26 was used to demonstrate the feasibility of sequencing. The test system of FIG. 26 is similar to the system of FIG. 5, but lacks a camera in the position of camera 410.

In the system of FIG. 26, a laser diode 2601 serves as a light source. Laser diode 2601 may, for example, transmit light at 650 nm, and the transmitted light may be filtered with an excitation filter. A prism 2602 (similar to prism 404) receives the light, and includes a gold-plated detection surface 2603 on which a flow cell and cover glass are placed. In the system of FIG. 26, prism 2602 is made of PMMA, and the gold plating on detection surface 2603 is 50 nm thick. A 10 megapixel camera 2604 images a plane within the flow cell. An emission filter 2605 may be present, to filter any fluorescent light emanating from the flow cell.

Detection surface 2603 was treated with streptavidin and incubated for four hours. A 30% biotinylated sequencing primer was applied to detection surface 2603, and incubated for 15 minutes at room temperature. Detection surface 2603 was washed with a low salt solution. About 10 μl of the nanoball solution was applied to detection surface 2603, and incubated for 30 minutes at room temperature. Detection surface 2603 was again washed with low salt solution, and Cy5 labeled dCTP was applied and incubated for five minutes at room temperature. In a first test, the Cy5 labeled dCTP was applied without Bsu polymerase, and in a second test, the Cy5 labeled dCTP was applied with Bsu polymerase. In each case, detection surface 2603 was then washed with a high salt solution (800 mM NaCl). Prism 2602 was then placed in the system of FIG. 26 for imaging.

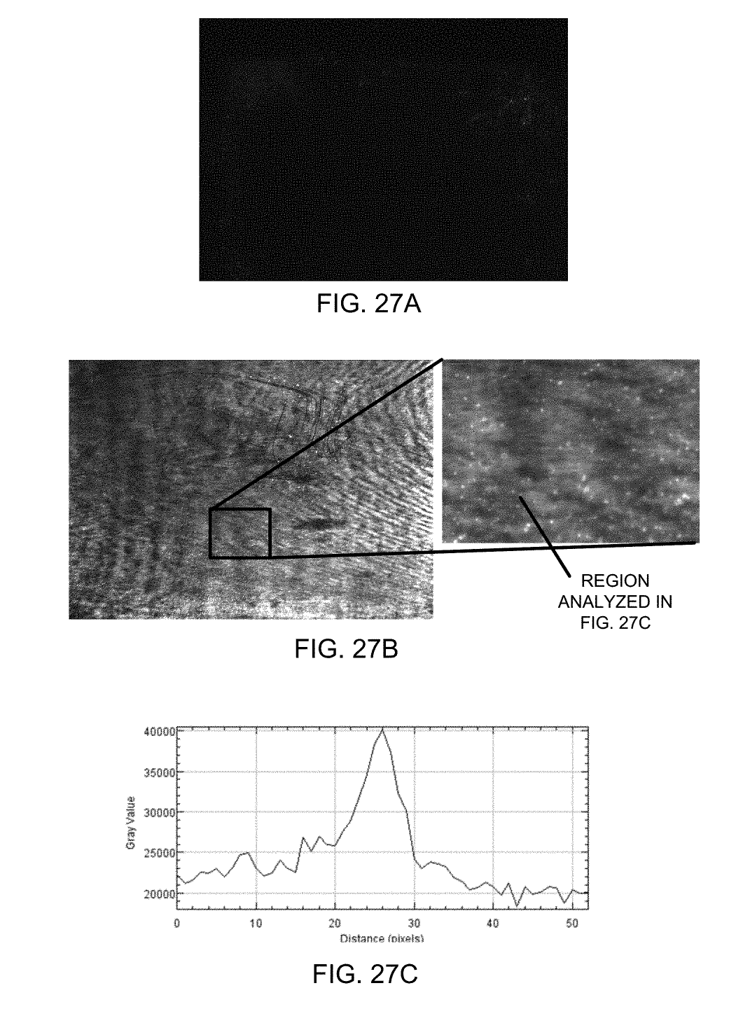

FIG. 27A illustrates a digital image taken by camera 2604 in the case that no Bsu polymerase was used. FIG. 27A is essentially a featureless dark rectangle.

FIG. 27B illustrates a digital image taken by camera 2604 in the case that Bsu polymerase was used. Significant lightening of the image as compared with FIG. 27A indicates the detection of significant fluorescence. An inset of FIG. 27B is magnified to show that fluorescence from individual nanoballs is resolved. FIG. 27C illustrates a digital “slice” (a graph of pixel intensity values along a line segment) taken across the image in the region of a particular nanoball, indicating a light intensity peak, which in turn indicates the binding of dCTP at that particular nanoball. In these examples, the exposure time of the camera was 3 seconds, and 5.82 dB of gain was applied.

Thus, nucleotide binding is detected at the individual nanoball level. A test such as that shown in FIGS. 27A-C may be sufficient for some applications. For example, sequencing of one base may be sufficient for a chromosome counting assay performed in non-invasive prenatal testing

Comparison of TIRF and SPEF

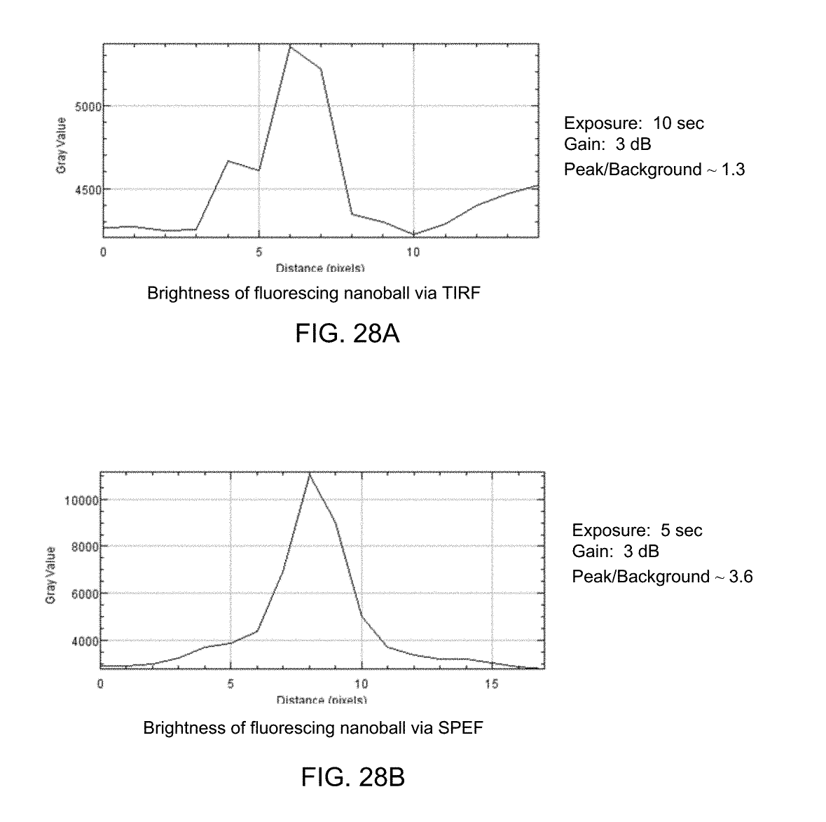

In another experiment, the system of FIG. 26 was used to compare the performance of total internal reflectance fluorescence (TIRF) imaging with surface plasmon enhanced fluorescence (SPEF) imaging, the difference in the two tests being that the prism used for SPEF was gold plated on its detection surface, while the prism used for TIRF did not have a gold layer. Samples were prepared and the fluorescence from a single nanoball in each sample was resolved and measured. FIG. 28A illustrates a digital “slice” (a graph of pixel intensity values along a line segment) taken across the TIRF image in the region of a particular nanoball. In the arbitrary units of FIG. 28A (resulting from a 10 second exposure with 3 dB gain applied), the particular imaged nanoball gave 5,340 digital counts of brightness, as compared with the brightness of the background field of about 4,200 digital counts. The ratio of the detected signal to the background (5,340/4,200) is about 1.3, and is an indication of the signal-to-noise ratio of the system, and of the ability of the system to reliably detect bindings.

By comparison, FIG. 28B is a digital slice taken across the SPEF image in the region of a particular nanoball. For the SPEF image, a five second exposure and 3 dB of gain were used. As is shown in FIG. 28B, the particular imaged nanoball gave 11,040 digital counts of brightness, as compared with the background brightness of about 3,000 digital counts. The ratio of the detected signal to the background (11,040/3,000) is about 3.6, indicating a much better signal-to-noise ration that in the TIRF image.

While a detailed description of presently preferred embodiments of the invention has been given above, various alternatives, modifications, and equivalents will be apparent to those skilled in the art without varying from the spirit of the invention. Therefore, the above description should not be taken as limiting the scope of the invention, which is defined by the appended claims.

It is to be understood that any workable combination of the features and capabilities disclosed above in the various embodiments is also considered to be disclosed. For example, any of the sensing modalities discussed above may be partially or fully incorporated into a disposable module, and any of the sensing modalities may be performed using a camera or other imaging optics, or may be performed using lensless contact imaging. The sensing modalities may be used in any workable combination, in any workable arrangement.

What is claimed is:

1. A system, comprising:
a computerized controller;
a prism having an input face, an output face, and a detection face, wherein, the detection face of the prism is plated with a metal;
a flow cell disposed at the detection face of the prism;
a plurality of reservoirs for holding respective fluids;
a plurality of valves connected respectively with the plurality of reservoirs;
a mechanism to generate fluid flow;
an illumination system positioned to direct light into the input face of the prism such that the light reaches the detection face of the prism; and
a sensing system positioned to image a plane in or adjacent the flow cell;
wherein the reservoirs, valves, mechanism to generate fluid flow, and flow cell are configured such that fluid from any particular one of the plurality of reservoirs can be individually supplied to the flow cell under the impetus of the mechanism to generate fluid flow and under control of the computerized controller, by opening of the respective valve of the particular reservoir and closing the other valves;
and wherein the prism, flow cell, plurality of reservoirs, and the plurality of valves are comprised in a disposable cartridge.

2. The system of claim 1, wherein the prism is a triangular prism.

3. The system of claim 1, wherein:
the prism is a trapezoidal prism having coplanar input and output faces, the detection face being parallel to and spaced apart from the input and output faces, the trapezoidal prism also having a first angled reflection face joining a first edge of the detection face with the input face and a second angled reflection face joining a second edge of the detection face with an edge of the output face; and
the illumination system is positioned to direct light into the input face of the trapezoidal prism such that the light reflects from the first angled reflection face of the trapezoidal prism and reaches the detection face of the trapezoidal prism.

4. The system of claim 1, wherein the detection face is patterned to enhance sensing of the face using the phenomenon of surface plasmon resonance.

5. The system of claim 1, wherein the sensing system performs surface plasmon resonance imaging or surface plasmon enhanced fluorescence imaging.

6. The system of claim 1, wherein the sensing system senses in a reflection mode.

7. The system of claim 1, wherein the sensing system senses in a transmission mode.

8. The system of claim 1, wherein the system images in multiple modes.

9. The system of claim 8, wherein the system performs both surface plasmon resonance imaging and surface plasmon enhanced fluorescence imaging.

10. The system of claim 1, wherein the flow cell is in the shape of a rectangle, and fluids enter the flow cell at one corner of the rectangle and exit the flow cell at the opposite corner of the rectangle.

11. The system of claim 1, wherein the flow cell is in the shape of a rectangle and has in input edge on one edge of the rectangle and an output edge at the opposite edge of the rectangle, the system further comprising:
a lead in channel for carrying fluids to the flow cell, the lead in channel being in the shape of a triangle having one edge joining the input edge of the flow cell, wherein fluids enter the lead in channel at the vertex of the triangle not adjacent to the input edge of the flow cell; and
a lead out channel for carrying fluids from the flow cell, the lead out channel being in the shape of a triangle having one edge joining the output edge of the flow cell, wherein fluids exit the lead out channel at the vertex of the triangle not adjacent to the output edge of the flow cell.

12. The system of claim 11, wherein the lead in channel is perpendicular to the flow cell.

13. The system of claim 11, wherein the lead out channel is perpendicular to the flow cell.

14. The system of claim 11, wherein the lead in channel and the lead out channel are of a constant cross section.

15. The system of claim 11, wherein the lead in channel, the lead out channel, or both the lead in channel and the lead out channel have a varying cross section

16. The system of claim 1, wherein the light source maintains an constant angle of incidence relative to the input face of the prism.

17. A cartridge, comprising:
a housing defining a plurality of reagent reservoirs and a sample reservoir;
a flow cell;
a prism having an input face, and output face, and a detection face; and
a plurality of valves connected respectively with the plurality of reservoirs and connected with the flow cell such that fluid from any particular one of the reservoirs can be individually supplied to the flow cell by opening of the respective valve of the particular reservoir and closing the other valves.

18. The cartridge of claim 17, wherein the detection face is patterned to enhance sensing of the face using the phenomenon of surface plasmon resonance.

19. The cartridge of claim 17, further comprising at least one waste well for receiving any fluid exiting the flow cell.

20. The cartridge of claim 17, wherein the prism is a trapezoidal prism having coplanar input and output faces, a detection face parallel to and spaced apart from the input and output faces, the trapezoidal prism also having a first angled reflection face joining a first edge of the detection face with the input face and a second angled reflection face joining a second edge of the detection face with an edge of the output face, wherein the input and output faces are accessible from outside the housing.

21. The cartridge of claim 17, further comprising a detection system, wherein the detection system includes a light source and an array light sensor, and wherein the detection system further includes a nanohole array, and the detection system detects effects of light reaching the flow cell via extraordinary optical transmission through the nanohole array.

22. The cartridge of claim 21, wherein the detection system performs surface plasmon resonance imaging or surface plasmon enhanced fluorescence imaging.
